(19) United States
(12) Patent Application Publication
(10) Pub. No.: US 2024/0108404 A1
(43) Pub. Date: Apr. 4, 2024

(54) DEVICES, METHODS AND SYSTEMS FOR RENAL DENERVATION

(71) Applicant: Otsuka Medical Devices Co., Ltd., Tokyo (JP)

(72) Inventors: Andrew Weiss, San Francisco, CA (US); Liang Zhai, Belmont, CA (US); Dimitri Augustin, San Mateo, CA (US); Andrea Methven, Happy Valley, OR (US)

(21) Appl. No.: 18/481,220

(22) Filed: Oct. 4, 2023

Related U.S. Application Data
(60) Provisional application No. 63/378,328, filed on Oct. 4, 2022.
(60) Provisional application No. 63/578,123, filed on Aug. 22, 2023.

(51) Int. Cl. A61B 18/14

(52) U.S. Cl. CPC A61B 18/1492; A61B 2018/00434

(57) ABSTRACT

A technique of renal denervation comprising methods and devices for generating one or more bubbles to at least partially fragment a calcification of a calcified region of a renal artery and for ablating one or more nerves about, within, or surrounding the calcified region of the renal artery after the calcification has been at least partially fragmented. Other embodiments are described and claimed.

This application claims the benefit of priority of U.S. Provisional Patent Application No. 63/378,328, filed on Oct. 4, 2022, titled “CATHETER HAVING PRETREATMENT AND TREATMENT UNIT AND METHOD OF USE,” and U.S. Provisional Patent Application No. 63/578,123, filed on Aug. 22, 2023, titled “CATHETER HAVING PRETREATMENT AND TREATMENT UNIT AND METHOD OF USE,” which are incorporated herein by reference in their entirety to provide continuity of disclosure.

### BACKGROUND

### Field

This application relates generally to minimally-invasive apparatuses, devices, systems, and methods that provide energy delivery to a targeted anatomical location of a subject, and more specifically, to catheter-based, intraluminal devices and systems configured to deliver ultrasonic energy to treat tissue, such as nerve tissue.

### Background Information

High blood pressure, also known as hypertension, commonly affects adults. Left untreated, hypertension can result in renal disease, arrhythmias, heart failure, and stroke. In recent years, the treatment of hypertension has focused on interventional approaches to inactivate the renal nerves surrounding a renal artery. Autonomic nerves tend to follow blood vessels to the organs that they innervate. Intraluminal devices, such as catheters, may reach specific structures, such as the renal nerves, that are proximate to the lumens in which the catheters travel. Accordingly, catheter-based systems can provide renal denervation therapy within the lumens to inactivate the renal nerves surrounding the vessel walls.

### SUMMARY

The present disclosure is defined in the independent claims. Further embodiments of the present disclosure are defined in the dependent claims. Methods, apparatuses and systems are provided herein.

A tissue treatment system for renal denervation is provided, the tissue treatment system comprising a bubble generation device configured to generate one or more bubbles to at least partially fragment a calcification of a calcified region of a renal artery; and an ablation device configured to ablate one or more nerves about, within, or surrounding the calcified region of the renal artery after the calcification has been at least partially fragmented.

A tissue treatment system configured to ablate one or more nerves about, within, or surrounding a calcified region of a renal artery is provided, the tissue treatment system comprising a non-transitory computer readable memory storing instructions. The tissue treatment system further comprises one or more processors configured to execute the stored instructions to cause the tissue treatment system to detect calcification within the renal artery in a target area, and lower a default acoustic entry power setting and increase a duration of ablation setting based on the detected calcification.

A further tissue treatment system is provided, the tissue system comprising an ablation device configured to ablate one or more nerves innervating a kidney; a non-transitory computer readable memory storing instructions; and one or more processors configured to execute the stored instructions to cause the tissue treatment system to detect a calcification amount at one or more target locations along a main renal artery and at one or more target locations along at least one of an accessory renal artery or a renal artery branch; determine whether the calcification amount is lower at one or more target locations along a main renal artery or at one or more target locations along at least one of an accessory renal artery or a renal artery branch; and prompt a user, using a graphic user interface, to perform an ablation at the one or more target locations along the main renal artery or, alternatively, at the one or more target locations along at least one of the accessory renal artery or the renal artery branch based on whether the calcification score is lower at the one or more target locations along the main renal artery or at one or more target locations along at least one of the accessory renal artery or the renal artery branch.

A method of renal denervation is provided, the method comprising delivering a catheter to a calcified region of a body lumen wall, wherein the catheter includes a proximal balloon, a middle balloon, and a distal balloon mounted on a catheter shaft, and wherein the catheter includes a laser fiber disposed on a surface of the middle balloon. The method further comprises generating, by the laser fiber, one or more bubbles to at least partially fragment the calcified region of the body lumen wall and ablating one or more nerves about, within, or surrounding the calcified region of the body lumen wall after the calcified region has been at least partially fragmented.

A further method of renal denervation is provided, the method comprising delivering a catheter to a calcified region of a body lumen wall, wherein the catheter includes a barbell-shaped balloon mounted on a catheter shaft, and wherein the catheter includes a laser fiber disposed on a surface of a center region of the barbell-shaped balloon. The method further comprises inflating the barbell-shaped balloon such that the center region is longitudinally aligned with the calcified region, and the calcified region is longitudinally between a proximal region and a distal region of the barbell-shaped balloon; generating, by the laser fiber, one or more bubbles to at least partially fragment the calcified region of the body lumen wall and ablating one or more nerves about, within, or surrounding the calcified region of the body lumen wall after the calcified region has been at least partially fragmented.

A further method of renal denervation is provided, the method comprising delivering a catheter to a calcified region of a body lumen wall, wherein the catheter includes a proximal balloon and a distal balloon mounted on a catheter shaft, and wherein the catheter includes a laser fiber disposed on a surface of the proximal balloon; generating, by the laser fiber, one or more bubbles to at least partially fragment the calcified region of the body lumen wall; and ablating one or more nerves about, within, or surrounding the calcified region of the body lumen wall after the calcified region has been at least partially fragmented.

Some of the features in the present disclosure are broadly described in order that the section entitled Detailed Description is better understood and that the present contribution to the art is better appreciated. Additional features of the present disclosure are hereinafter described. In this respect, the present disclosure is not limited in its implementation to the details of the components or steps set forth herein or as illustrated in the several figures of the Drawing(s) and that the components or steps may be carried out in various ways. Also, the phraseology and terminology employed herein are for the purpose of the description and should not be regarded as limiting.

Also presented is a system for renal denervation, comprising a bubble generation device configured to generate one or more bubbles to at least partially fragment a calcification of a calcified region of a renal artery; and an ablation device configured to ablate one or more nerves about, within, or surrounding the calcified region of the renal artery after the calcification has been at least partially fragmented. The system may further comprise an imaging device. The system may further comprise a controller or controller system and a memory storing a computer program product comprising program code portions for performing the steps of any of the methods presented herein when the computer program is executed by the controller or controller system. The system may be configured as a catheter device, for example as an ablation catheter.

Also provided is computer program product comprising program code portions for performing the steps of any of the methods presented herein when the computer program is executed by a processing device. The processing device may be comprised by the controller or controller system.

### BRIEF DESCRIPTION OF THE DRAWINGS

The foregoing is a summary, and thus, necessarily limited in detail. The above-mentioned aspects, as well as other aspects, features, and advantages of the present technology are described below in connection with various embodiments, with reference made to the accompanying drawings.

FIG. 1 is a diagram illustrating a perspective view of a general ultrasound-based ablation system, in accordance with an embodiment of the present disclosure.

FIG. 2 is a diagram illustrating a detailed cut-away cross-sectional view of a distal end of the general ultrasound-based ablation system, as shown in FIG. 1, in accordance with an embodiment of the present disclosure.

FIG. 3 is a diagram illustrating a cut-away perspective view of a catheter device for pretreating and treating an anatomical structure, in accordance with an embodiment of the present disclosure.

FIG. 4A is a diagram illustrating a side view of a catheter device for pretreating and treating an anatomical structure, in accordance with an embodiment of the present disclosure.

FIG. 4B is a diagram illustrating a side view of a catheter device for pretreating and treating an anatomical structure, in accordance with an embodiment of the present disclosure.

FIG. 4C is a diagram illustrating a side view of a catheter device for pretreating and treating an anatomical structure, in accordance with an embodiment of the present disclosure.

FIG. 4D is a diagram illustrating a side view of a catheter device for pretreating and treating an anatomical structure, in accordance with an embodiment of the present disclosure.

FIG. 5 is a schematic diagram illustrating a catheter device for pretreating and treating an anatomical structure, in accordance with an embodiment of the present disclosure.

FIG. 6 is a flow diagram illustrating a method of fabricating a catheter device for pretreating and treating an anatomical structure, in accordance with an embodiment of the present disclosure.

FIG. 7 is a flow diagram illustrating a method of pretreating and treating an anatomical structure by way of a catheter device, in accordance with an embodiment of the present disclosure.

FIG. 8 is a flow diagram illustrating a method of pretreating and treating a renal artery, in accordance with an embodiment of the present disclosure.

FIG. 9A is a diagram illustrating a side view of a catheter device having a deflated proximal and distal balloon for blocking debris after pretreating a renal artery, in accordance with an embodiment of the present disclosure.

FIG. 9B is a diagram illustrating a side view of a catheter device having an inflated proximal and distal balloon for blocking debris after pretreating a renal artery, in accordance with an embodiment of the present disclosure.

FIG. 10 is a diagram illustrating a side view of a catheter device having a cone-shaped balloon at a distal portion for blocking debris after pretreating a renal artery, in accordance with an embodiment of the present disclosure.

FIG. 11 is a diagram illustrating a side view of a catheter device having a barbell-shaped balloon for blocking debris after pretreating a renal artery, in accordance with an embodiment of the present disclosure.

FIG. 12 is a diagram illustrating a side view of one embodiment of a catheter device having a scoop for collecting debris after pretreating a renal artery, in accordance with an embodiment of the present disclosure.

FIG. 13 is a diagram illustrating a main renal artery and an accessory renal artery running parallel to the main renal artery.

FIG. 14 is a diagram illustrating a main renal artery and a renal artery branch running nonparallel to the main renal artery.

FIG. 15 illustrates example details of a controller, in accordance with an embodiment.

FIG. 16 is a diagram illustrating a cross section of a transducer, in accordance with an embodiment of the present disclosure.

The illustrated embodiments are merely examples and are not intended to limit the disclosure. The schematics are drawn to illustrate features and concepts and are not necessarily drawn to scale.

### DETAILED DESCRIPTION

The foregoing is a summary, and thus, necessarily limited in detail. The above-mentioned aspects, as well as other aspects, features, and advantages of the present technology will now be described in connection with various embodiments. The inclusion of the following embodiments is not intended to limit the disclosure to these embodiments, but rather to enable any person skilled in the art to make and use the contemplated invention(s). Other embodiments may be utilized, and modifications may be made without departing from the spirit or scope of the subject matter presented herein. Aspects of the disclosure, as described and illustrated herein, can be arranged, combined, modified, and designed in a variety of different formulations, all of which are explicitly contemplated and form part of this disclosure.

Any system, apparatus, device, product-by-process, composition of matter, process, technique, or method, herein described, is useful in the health, medical, or surgical fields, including oncological care, procedures, and surgeries; however, the subject matter of the present disclosure may extend, or apply, to other conditions or fields of health, medicine, or surgery; and such extensions or implementations are encompassed by the present disclosure. Any system, apparatus, device, product-by-process, composition of matter, process, technique, or method, herein described, encompasses technologies that are applicable to health, medical, or surgical procedures for any other anatomical region that will benefit from the use of a catheter to facilitate access to an interior of an animal body, such as a human body.

Various systems, apparatuses, devices, products-by-process, compositions of matter, processes, techniques, or methods may be below-described; and when described, provide examples thereof, in accordance with embodiments of the present disclosure. None of the below-described embodiments limits any claimed embodiment; and any claimed embodiment may also encompass systems, apparatuses, devices, products-by-process, compositions of matter, processes, techniques, or methods which may differ from below-described examples, but are also encompassed by the present disclosure. The claimed embodiments are not limited to any one or any combination of any below-described system, apparatus, device, product-by-process, composition of matter, process, technique, or method.

Furthermore, this Detailed Description sets forth numerous specific details in order to provide a thorough understanding of the various embodiments described throughout the present disclosure; however, the herein described embodiments may be practiced without these specific details. In other instances, well-known methods, techniques, procedures, or components have not been described in detail so as not to obscure the herein described embodiments.

Approximately a third of patients are typically insufficiently responsive or non-responsive to renal denervation therapy, e.g., having less than approximately 5-mm mercury (Hg) reduction in daytime ambulatory systolic blood pressure (dASBP). Calcification or calcium deposits involving renal arteries may be a cause.

Calcification includes calcium buildup or calcium deposits that may increase arterial stiffness and be a secondary cause of hypertension. Although the calcification may not be sufficiently significant to act as a secondary cause of hypertension, it may nevertheless interfere with renal denervation procedures. Calcification within an arterial wall of a renal artery may interfere with sonication by ultrasound therapy devices as ultrasound signals tend to reflect from calcium deposits. Similarly, in using radio-frequency (RF) devices, conductive heating required for treating nerves associated with renal structures is adversely insulated by calcification of an arterial wall. Also, by using RF devices, obtaining proper apposition of an electrode against a blood vessel wall may be difficult due to calcification thereof. In addition, the calcification may reshape a lesion, thereby leading to inconsistent ablations and treatment.

As such, there is a need for a method of imaging a calcified region, fragmenting at least a portion of the imaged calcified region, and ablating tissue surrounding the imaged calcified region. Similarly, there is a need for a device for use in performing the method.

To address at least the foregoing challenges, the present disclosure generally involves a catheter device and methods for pretreating and treating an anatomical structure, such as renal arteries, to treat a medical condition. The present disclosure involves a catheter and methods for fragmenting calcification from calcified regions and ablating the nerves surrounding the renal artery to treat a medical condition. Further, the present disclosure involves a catheter device and methods for identifying calcified regions and verifying that the regions have been removed prior to ablation therapy.

Renal artery calcification (also known as renovascular calcification) refers to mineral, e.g., calcium, depositions detected on or in the walls of the renal arteries (or their branches). One is considered to have renal artery calcification when the density of the mineral depositions detected is ≥130 Hounsfield units on CT.

The cause of renal artery calcification is generally a result of the conversion of vascular smooth muscle cells of osteoblasts, due to retention of phosphate, hypercalcemia, previous dialysis treatment, active vitamin D administration or calcification inhibitor deficiency among other causal factors.

Calcifications may be present at the intima, the media or both layers of the renal arteries. In atherosclerosis, the intima becomes greatly inflamed, thickened and calcified. However, predominately medial artery calcification is associated with hypertension, diabetes mellitus and/or chronic kidney disease. Medial calcification is associated with stiffened arterial wall, cardiovascular events and death.

To assess the degree and/or severity of the calcification, the peripheral arterial calcium scoring system (PACSS) can be used: grade 0: no visible calcifications at the referred region; grade 1: unilateral calcifications less than 5 cm in maximum diameter; grade 2: unlilateral calcification equal to, or greater than 5 cm in maximum diameter; grade 3: bilateral calcification less than 5 cm in maximum diameter; grade 4: bilateral calcification equal to, or greater than, 5 cm in maximum diameter. Calcified renal arteries can be firm, dense, and tubular, with solid white plaques.

Imaging of renal artery calcification can be done using x-rays, ultrasonography and non-contrast CT, PET scans, extravascular ultrasound, intravascular ultrasound, MRI, or angiography. The appearance of calcification using ultrasound is that of a hyperechoic foci accompanied by acoustic shadowing.

Calcified regions may be found in the wall of the renal artery and extend over some longitudinal distance of the renal artery. Calcified regions may also be found within the lumen of the arterial wall. Calcified regions can require high pressures (sometimes as high as 10-15 or even 30 atmospheres) to fragment. The calcified region may create varying distances from an ablation transducer or an ablation electrode of an ablation catheter to the nerves. As a result, renal denervation may not be as uniformly effective across the extent of the calcified region.

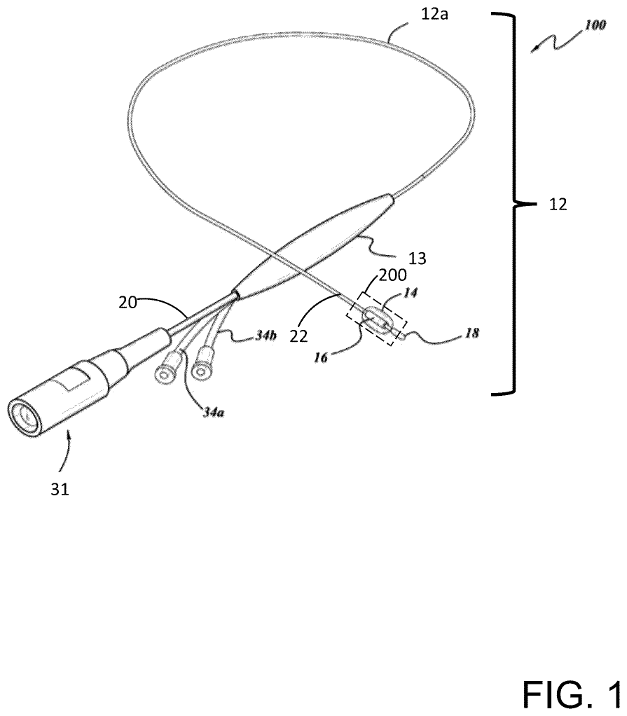

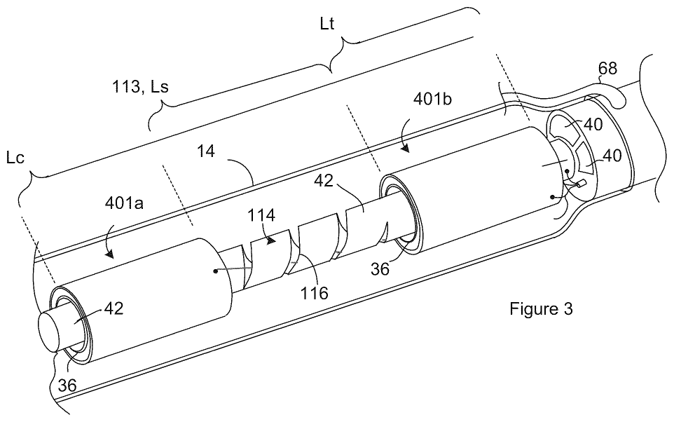

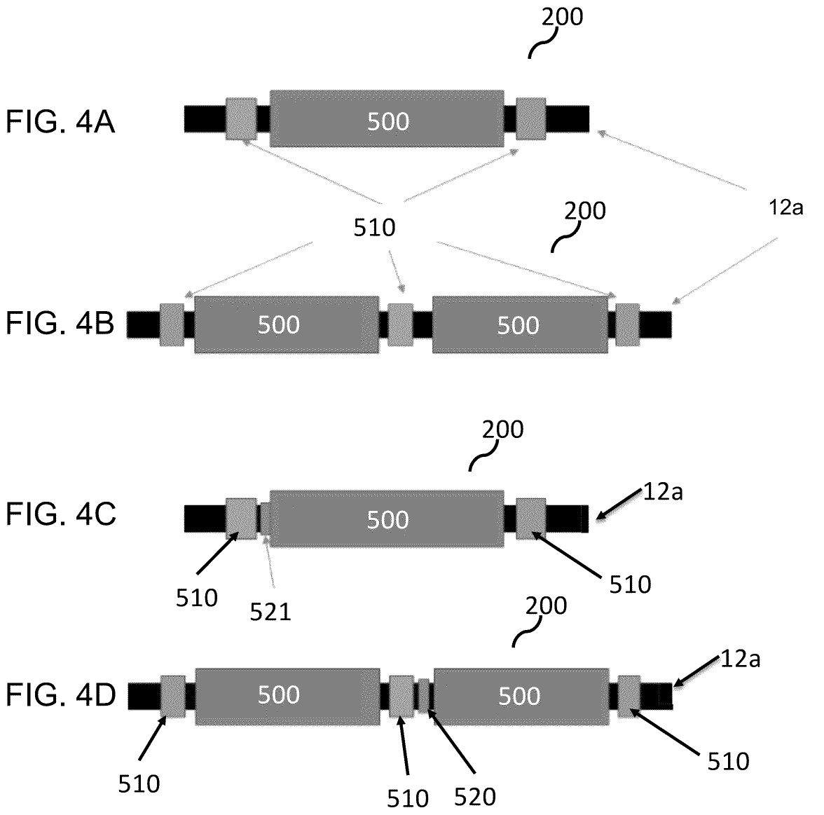

Referring to FIG. 1, this diagram illustrates, in a perspective view, a general ablation system, in accordance with an embodiment of the present disclosure. A system 100 may be implementable with any embodiment, or embodiments of the present disclosure, such as a catheter device, e.g., a catheter device 200 (FIGS. 3A-7). The system 100 comprises a catheter 12 having a proximal end 20 and a distal end 22. The catheter 12 comprises a catheter shaft 12a, an expandable member 14, e.g., a balloon or a medical balloon, and a tip member 18. The system 100 further comprises an electrical coupler 31 disposable at a proximal end of the catheter shaft 12a and configured to couple the system 100 with a power source or a power generator (not shown). The power generator may be a source of high voltage pulses for decalcification electrodes (FIGS. 4A-7). There can be a high voltage switch (not shown) that can be set to control the duration of the pulse. The pulse duration depends on the surface area of the decalcification electrodes and is sufficient to generate a gas bubble at the surface of the decalcification electrode to cause a plasma arc of electric current to jump the bubble and create a rapidly expanding and collapsing bubble, which creates the mechanical shock wave in the expandable member 14. The shock wave can be as short as a few microseconds. A shock wave is a very large localized pressure perturbation. A shock wave often has a pressure of a few hundred or thousand bars, and has a pulse-like pressure profile. In some embodiments, the system 100 further comprises a catheter device (e.g., catheter device 200 of FIGS. 3A-7). The catheter device can comprise at least one unit, each at least one unit comprising at least one ablation device, for example, a transducer 16, e.g., at least one ultrasound transducer, disposable within the expandable member 14.

As described below, the system 100 may also include alternative components to generate bubbles and/or shock waves to break up calcification. The system 100 can include a laser source to generate and transmit a laser to the treatment area. The laser can be a pulsed laser that includes pulses of light spaced in time. Energy from the laser can be absorbed by fluid at the treatment site, and a vapor microbubble can form as a result. The microbubbles collapse to create the mechanical shock wave that breaks up calcification.

Still referring to FIG. 1, the expandable member 14 is disposable between the catheter shaft 12a and the tip member 18. The expandable member 14, e.g., a balloon, is further configured for disposition along the distal end 22 of the catheter 12. The expandable member 14 comprises at least one of: a medically compliant balloon, a medically semi-compliant balloon, and a medically noncompliant balloon. The expandable member 14 comprise at least one material of: a nylon, a polyimide, a polyimide film, a thermoplastic elastomer, e.g., a PEBAX™ thermoplastic elastomer, a medical-grade thermoplastic polyurethane elastomer, e.g., a PELLETHANE™ thermoplastic polyurethane elastomer, a pellethane, an isothane, and any other suitable polymer or any combination thereof.

Still referring to FIG. 1, the catheter 12 comprises a handle 13 disposable near, or at, the proximal end of the catheter shaft 12a. The system 100 comprises at least one electrical coupler 31 configured to couple the catheter system 100 with at least one external electrical conductor (not shown), the at least one external electrical conductor in electrical communication with external electronics (not shown). The at least one external electrical conductor comprises at least one of: at least one wire, at least one cable, and at least one flexible printed circuit (FPC).

Still referring to FIG. 1, the catheter 12 comprises at least one lumen, e.g., at least one electrical lumen (not shown). Each at least one electrical lumen extends from the at least one electrical coupler 31, along a longitudinal length of the catheter shaft 12a toward a distal end of the catheter shaft 12a. Each of the at least one electrical lumen is configured to accommodate and retain at least one conductor carrier (not shown), the at least one conductor carrier configured to carry the at least one electrical conductor. The at least one electrical conductor can be in electrical communication with the external electronics through the at least one electrical coupler 31 and the at least one external electrical conductor. When an electrical conductor carrier carries several electrical conductors, the electrical conductor carrier comprises an electrically insulating jacket (not shown). When an electrical conductor comprises a single electrical conductor, the electrical conductor comprises an electrical insulator disposed on the electrical conductor, whereby the electrical insulator serves as an electrical conductor carrier.

Still referring to FIG. 1, the handle 13 optionally comprises at least one fluid port, e.g., fluid ports 34a, 34b, for coupling the catheter 12 with a conduit (not shown). Suitable conduits comprise at least one of: a tube, a hose, and any hollow member. The conduit facilitates fluid communication between the at least one fluid port and a fluid source (not shown). The fluid source comprises at least one of: a pump, a tank, a reservoir, and a vessel. The catheter shaft 12a may comprise at least one fluid lumen (not shown). Each at least one fluid lumen facilitates fluid communication with each at least one fluid port and is disposable along a length of the catheter shaft 12a toward a distal end of the catheter shaft 12a. Further, several lumens, can be in fluid communication with corresponding pumps or other fluid transfer devices (not shown) via the ports 34a, 34b, e.g., a luer fitting, other standard, or non-standard, couplings, and the like.

Still referring to FIG. 1, the handle 13 optionally comprises at least one guidewire port (not shown) configured to receive at least one guidewire (not shown). The catheter shaft 12a optionally comprises at least one guidewire lumen (not shown) configured to accommodate and retain the at least one guidewire. The at least one guidewire lumen extends along a length of the catheter shaft 12a toward a distal end 22 of the catheter shaft 12a. Each at least one guidewire lumen facilitates fluid communication with each at least one guidewire port such that each at least one guidewire, inserted into each at least one guidewire port, can be received within each at least one guidewire lumen.

Still referring to FIG. 1, in some embodiments, each at least one lumen is operable as at least one of: a fluid lumen, a fluid conduit, a cable lumen, an electrical cable passageway, a guidewire lumen, and the like. For example, the cable lumen is shaped, sized, and otherwise configured, to receive an electrical cable (not shown), e.g., a coaxial cable, a wire, and any other electrical conductor. The electrical cable is configured to permit an electrode of an ultrasound transducer of the system 100 to be selectively activated to emit acoustic energy to a target of a subject.

Still referring to FIG. 1, in some embodiments, the fluid lumen is configured to transfer cooling fluid, e.g., water, a saline solution, and any other coolant liquid or gas, to, and from, the expandable member 14 disposed at the distal end 22 of the catheter shaft 12a. In some embodiments, the catheter shaft 12a comprises at least two fluid lumens, one for delivering cooling fluid to the expandable member 14 and the other for returning the cooling fluid from the expandable member 14. However, the catheter shaft 12a comprises at least one fluid lumen as desired or required. The at least one fluid lumen is disposable along any part of the catheter shaft 12a, e.g., along the center-line, offset from the center-line, etc., and/or can include any cross-sectional shape, e.g., circular, oval, rectangular or other polygonal, irregular, etc., as desired or required.

In some embodiments, the catheter device 200 comprises a tissue collection unit (not shown) or a scraping unit (not shown). The scraping unit is located adjacent to the tissue collection unit, which allows the calcification debris (also referred to as calcification dust or calcification fragments), e.g., after cavitation or as an alternative to cavitation, to be scraped and collected by the tissue collection unit. For example, in certain embodiments where the calcification is located in the lumen of the blood vessel itself, cavitation may not be necessary, and the calcium may be scraped or otherwise removed from the vessel wall without the need for cavitation. In certain embodiments, calcification in the intima and/or within the blood vessel lumen may be cavitated using a shock wave source, and the calcification debris, e.g., cracked calcification, may be removed using the tissue collection unit and/or scraping unit and/or a vacuum catheter. In an embodiment, the scraping unit can be located between the tip member 18 and the expandable member 14. In another embodiment, the scraping unit can be located at the proximal end of the expandable member 14. The scraping unit can be a scoop-shaped cutting blade mounted on the catheter device. In another embodiment, the scraping unit can be a rotary tissue borer. The tissue collection unit comprises an opening for the calcification fragments/debris, to enter using a vacuum-like suction or natural momentum for removal from the bodily lumen. In an embodiment, the tissue collection unit is located behind the scraping unit so that the scraped calcification debris can flow into the collection unit after being scraped from the bodily lumen.

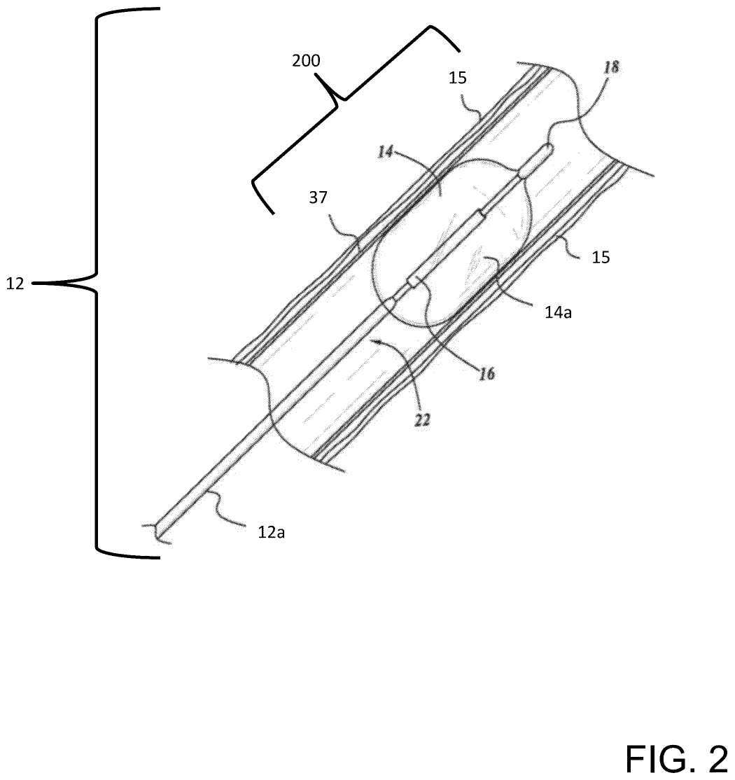

Referring to FIG. 2, this diagram illustrates, in a detailed cut-away cross-sectional view, the distal end of the general catheter system 100, comprising the expandable member 14, as shown in FIG. 1, in accordance with an embodiment of the present disclosure. Alternatively, the device 200 (FIGS. 3-7) comprises the expandable member 14, rather than does the system 100. The at least one unit, comprising at least one transducer 16, e.g., at least one ultrasound transducer, is configured to be disposable in at least one location, for example inside the expandable member 14 (e.g., in an interior cavity 14a of the expandable member 14), or outside the expandable member 14, as desired or required by a given implementation. In some embodiments, when expanded, the outer wall of the expandable member 14 is generally parallel with the walls of the at least one transducer 16, e.g., the at least one transducer 16 comprises a cylindrical configuration. When inflated, the expandable member 14 at least partially surrounds the transducer 16 and at least partially contacts an adjacent wall 37 of a vessel, e.g., a blood vessel. The adjacent wall 37 of the blood vessel may be disposed adjacent nervous tissue 15.

Still referring to FIG. 2, in some embodiments, however, one or more portions of the expandable member 14 are configured to avoid contacting the adjacent wall 37 of the blood vessel when expanded. In some embodiments, the transducer 16 is liquid cooled along both its outer and inner electrodes, whereby cooling liquid, entering the expandable member 14, is permitted to pass both an exterior surface and an interior surface of the transducer 16 to transfer heat away from the transducer 16. In some embodiments, the cooling liquid or other fluid directly contacts an exterior surface and an interior surface of the transducer 16. The transducer 16 can include a reflective interface (not shown), e.g., along its interior surface, to permit ultrasonic energy, generated at the inner electrode, e.g., along the interior surface of the cylindrical transducer, to be radially reflected.

Referring to FIG. 3, illustrating a cut-away view, a catheter device 200, comprising a plurality of units 401a,b. In some embodiments, the catheter device 200 comprises three or more units. In certain embodiments, the plurality of units 401a,b comprise a cavitation transducer unit 401a and an ablation transducer unit 401b at a distal end 22 (FIG. 1) of the catheter shaft 12a. Each of the cavitation transducer unit 401a and the ablation transducer unit 401b comprise a transducer 16. Alternatively, cavitation and ablation can be provided for using separate catheters, one catheter having a cavitation transducer unit 401a and another catheter having an ablation transducer unit 401b. In certain embodiments having both a cavitation transducer unit 401a and an ablation transducer unit 401b on one catheter 200, the cavitation transducer unit 401a is located proximal to the ablation transducer unit 401b on the catheter 200. After the cavitation procedure, the catheter 200 is then pulled proximally to the decalcified area, and the ablation procedure may be performed. Moving the catheter distal to proximal (i.e., away from the kidney and toward the aorta) may help prevent/minimize mechanical trauma to the renal artery. In certain embodiments, an imaging device is used to determine whether the cavitation procedure sufficiently decalcified the area, e.g., sufficiently lowered the calcium score (e.g., the calcium score is 50 or below after cavitation) and/or the media-adventitia border is visible using, e.g., intravascular ultrasound imaging at a frequency of 20-50 MHz, e.g., 40 MHz.

Alternatively, the cavitation transducer unit 401a may be located distal to the ablation transducer unit 401b on the catheter 200 and the catheter may be pushed proximal to distal. This may allow ablation of a proportionally larger number of afferent renal nerves, which may be responsible for messaging the brain regarding pain during the ablation procedure. By ablating these nerves first, further ablation closer to the kidneys may be perceived as less painful by the patient.

The cavitation transducer unit 401a and ablation transducer unit 401b may be optimized for their respective functions. The wavelength and power of the cavitation transducer unit 401a can be optimized to generate cavitation within the blood that preferentially shatter calcification in the lumen, intima, and/or media of the blood vessel based upon inelasticity of the calcified region while leaving healthy, elastic blood vessel tissues unaffected. In certain embodiments, the cavitation transducer unit 401a has an operating frequency of 400 kHz to 3 MHz, e.g., 1-2 MHz or less than 3 MHz. The cavitation transducer unit 401a may deliver ultrasound at a temporal-average intensity output below 50 W/cm², e.g., 2 to 20 W/cm² to create shock waves at the calcification in order to fragment it. In certain embodiments, 10 to 100 short acoustic pulses 10 microseconds to 1 millisecond in duration separated by 20 milliseconds to 2 seconds (0.5 Hz to 50 Hz pulse repetition rate) each are generated until the calcification is fragmented. In some embodiments, a vacuum catheter is used to remove the debris.

In some embodiments, the ablation transducer unit 401b is optimized to ablate nerve, while the balloon 14 (FIG. 2) helps protect non-target tissue within the blood vessel wall from thermal injury, e.g., stenosis. In certain embodiments, liquid flows through the balloon 14 at a rate of 10 ml/min to 45 ml/min, during ablation therapy. In certain embodiments, balloon 14 is compliant. The inflation pressure can correspond to a flowrate of fluid circulated through the interior of the balloon 14. For example, the fluid may be circulated at a flowrate of 15 to 35 mL/min (e.g., 25 to 35 mL/min) to inflate the balloon 14 to the inflation pressure of 10 psi, which results in a first inflation diameter of 3 to 6 mm (e.g., 3.5 to 6 mm). When the compliant balloon 14 is inflated to a second inflation pressure of 30 psi, the balloon has a second inflation diameter of 8 mm to 9 mm. The inflation pressure can correspond to a flowrate of fluid circulated through the interior of the balloon 114 between an inlet channel and an outlet channel (not shown). For example, the fluid may be circulated at a flowrate of 35 to 50 mL/min (e.g., 40 to 45 mL/min) to inflate the balloon 14 to the inflation pressure of 30 psi, which results in the first inflation diameter of 8 to 9 mm. For example, the balloon can have the second inflation diameter of 8 mm at a second inflation pressure of 30 psi and a flowrate of 40 to 45 mL/min.

In certain embodiments, the flow is stagnant or low flow, e.g., less than 2 ml/min during calcium cavitation. Accordingly, flow may be lower during calcium cavitation, as compared to during ablation therapy.

In certain embodiments, the ablation transducer unit 401b emits unfocused ultrasound energy. The term unfocused, as used herein, refers to an ultrasonic energy beam that does not increase in intensity in the direction of propagation of the beam away from the transducer. In some embodiments, the ablation transducer unit 401b is an air-backed ablation transducer. In some embodiments, the ablation transducer unit 401b is a water-backed transducer. In certain embodiments, the ablation transducer unit 401b has an operating frequency of 7 MHz-20 MHz, e.g., 8.5 MHz to 15 MHz, or 8.5 to 9.5 MHz or 8.5 MHz to 13 MHz. In an embodiment, energizing the ablation transducer unit 401b may occur for a period between 5 seconds to 20 seconds. In an embodiment, energizing the ablation transducer unit 401b may occur for a period between 6 seconds to 10 seconds. In an embodiment, energizing the ablation transducer unit 401b may be done for a period of approximately 7 seconds. The amount of acoustic energy emitted by the ablation transducer unit 401b that enters target tissue surrounding the body lumen in which ablation transducer unit 401b is located is equal to an Acoustic Entry Power multiplied by a duration (7) that the acoustic signal is emitted. The Acoustic Entry Power is based on (and may be dependent on) various factors, including an output power level setting of the ablation unit excitation source (e.g., 1518a), a power efficiency of the system (including the components thereof), a frequency of the acoustic signal emitted by the ablation transducer unit 401b, a duration (7) of the acoustic signal emitted by the ultrasound transducer, and an amount of attenuation caused by a medium that is between the ultrasound transducer and the body lumen wall. Where the ablation transducer unit 401b is located within a balloon 14 through which a cooling fluid (e.g., water, sterile water, saline, or D5 W) is circulated, the cooling fluid and the balloon material (and potentially, any folds in the balloon material) are the medium between the ablation transducer unit 401b and the body lumen wall. Where the catheter is balloonless (i.e., devoid of a balloon), then blood traveling through the body lumen is the medium between the ultrasound transducer and the body lumen wall. The catheter can include a centering mechanism configured to generally center the ultrasound transducer within the body lumen. In certain embodiments, the centering mechanism is provided by a compliant balloon. Alternatively, or additionally, the centering mechanism can comprise one or more flexible acoustically transparent baskets attached to a catheter shaft (e.g., 212), or other structures, such as the spiral springs, but is not limited thereto.

In other words, the total energy absorbed (E_{eff}) in the targeted patient tissue (also referred to as the target tissue, the target zone, or the targeted region) surrounding a body lumen (within which the ablation transducer unit 401b is positioned) is the product of an Entry Acoustic Power (E₀) multiplied by a portion (β) (e.g., percent) of the energy used for ablation in the targeted region, the portion (β) (e.g., percent) of energy used for ablation in the targeted region is dependent on the extent of attenuation caused by the medium between the ultrasound transducer and the body lumen wall. More specifically, E_{eff}=β·E₀=(1−e^{-2afd})P₀T, where a is the attenuation coefficient (neper/MHz/cm), β the portion (e.g., percent) of energy used for ablation in the targeted region, f is the acoustic frequency, and d is the desired outer lesion boundary (also referred to as the lesion depth). The total energy absorbed (E_{eff}) in the targeted region can also be referred to herein as the effective energy (E_{eff}).

The Acoustic Entry Power (E₀) is the total acoustic power delivered into patient tissue, e.g., through a balloon wall. As the acoustic waves propagate through patient tissue, acoustic energy is attenuated and converted into heat, which results in a temperature increase in the tissue. Only a portion (β) (e.g., percent) of the Acoustic Entry Power (E₀) is absorbed by the targeted region, while the residual portion travels further and is absorbed by untargeted patient tissue beyond the targeted region. As a reminder, Energy is the product of power multiplied by time (duration). Thus, the effective energy (E_{eff}), which is the portion of acoustic energy absorbed by the targeted region, is equal to the product of β multiplied by the Acoustic Entry Power (E₀), i.e., E_{eff}=β·E₀, as was noted above. The value of β can depend on various different parameters, such as, but not limited to, an acoustic frequency and a desired lesion depth d.

To keep (maintain) the same lesion boundary d (lesion depth), E_{eff} should be kept constant. This statement is true when the treatment time (duration) T doesn't change significantly, and when an impact from heat conduction does not change significantly. It is noted that more total energy or effective energy is generally required if the treatment time (duration) T is increased significantly to compensate for the heat loss due to conduction. Table 3 below shows the Acoustic Entry Power for various different ultrasound frequencies, assuming a desired lesion depth (d) of 4 mm, and a treatment duration (7) of 7 seconds.

TABLE 3

| d = 4 mm, T = 7 seconds | | | | |
|---|---|---|---|---|
| α = 0.058 neper/Mhz/cm | 7 MHZ | 9 MHZ | 12 MHZ | 15 MHZ |
| β | 27.73% | 34.14% | 42.70% | 50.14% |
| Acoustic Entry Power (E₀) | 32.0 W | 26.0 W | 20.8 W | 17.7 W |
| | | (reference | | |
| | | value for | | |
| | | others) | | |

Table 4 below shows the Acoustic Entry Power for various different ultrasound frequencies, assuming a desired lesion depth (d) of 6 mm, and a treatment duration (7) of 7 seconds. As can be appreciated from a comparison between Table 4 and Table 3, a higher Acoustic Entry Power of 35.6 W should be used when there is a desire to produce a lesion depth of 6 mm, compared to an Acoustic Entry Power of 26.0 W that may be used where the desired lesion depth is 4 mm (as may be appropriate more distal, i.e., closer to the kidneys).

TABLE 4

| d = 6 mm, T = 7 seconds | | | | |
|---|---|---|---|---|
| α = 0.058 neper/Mhz/cm | 7 MHZ | 9 MHZ | 12 MHZ | 15 MHZ |
| β | 38.57% | 46.55% | 56.62% | 64.80% |
| Acoustic Entry Power (E₀) | 41.8 W | 34.6 W | 28.4 W | 24.9 W |
| | | (reference | | |
| | | value for | | |
| | | others) | | |

Table 5 below shows the Acoustic Entry Power for various different ultrasound frequencies, assuming a desired lesion depth (d) of 6 mm, and a treatment duration (7) of 10 seconds. As can be appreciated from a comparison between Table 5 and Table 4, a lower Entry Power of 24.2 W should be used when there is a desire to produce a lesion depth of 6 mm where the duration that the Acoustic Entry Power is delivered is 10 seconds, compared an Acoustic Entry Power of 36.4 W that may be delivered for a shorter duration of 7 seconds to produce the same desired lesion depth of 6 mm.

TABLE 5

| d = 6 mm, T = 10 seconds | | | | |
|---|---|---|---|---|
| α = 0.058 neper/Mhz/cm | 7 MHZ | 9 MHZ | 12 MHZ | 15 MHZ |
| β | 38.57% | 46.55% | 56.62% | 64.80% |
| Acoustic Entry Power (E₀) | 29.2 W | 34.6 W * | 19.9 W | 17.4 W |
| | | 7/10 = 24.2 W | | |
| | | (reference | | |
| | | value for | | |
| | | others) | | |

In certain such embodiments, a frequency of the acoustic energy is about 9 MHz, the duration of the acoustic power delivery is about 7 seconds, and the Acoustic Entry Power is about 34.6 W.

In some embodiments, calcium is within the media of the blood vessel and may be difficult to sufficiently fragment and/or remove. In some embodiments, the decalcification operation is omitted despite the presence of calcification. In order to compensate for calcification, detected by a user and/or the processor based on a calcium score and/or an indication that the media-adventitia border cannot be delineated or is unclear, a generator may lower the Acoustic Entry Power, while increasing the duration of the ablation and increasing the flow rate and/or decreasing the temperature of the cooling fluid. For example, the Acoustic Entry Power may be lowered by 30% below a default setting (i.e., a setting used in the absence of calcification and/or a setting not specifically set to compensate for calcification) while the treatment duration (7) is increased 35-45% above a default setting. In some embodiments, the flow rate is increased, e.g. by 5% or more above a default setting. The increased flow rate will remove power from the system that must be compensated for by the system by increasing the treatment duration (7) disproportionately (i.e., more than) to the Acoustic Entry Power.

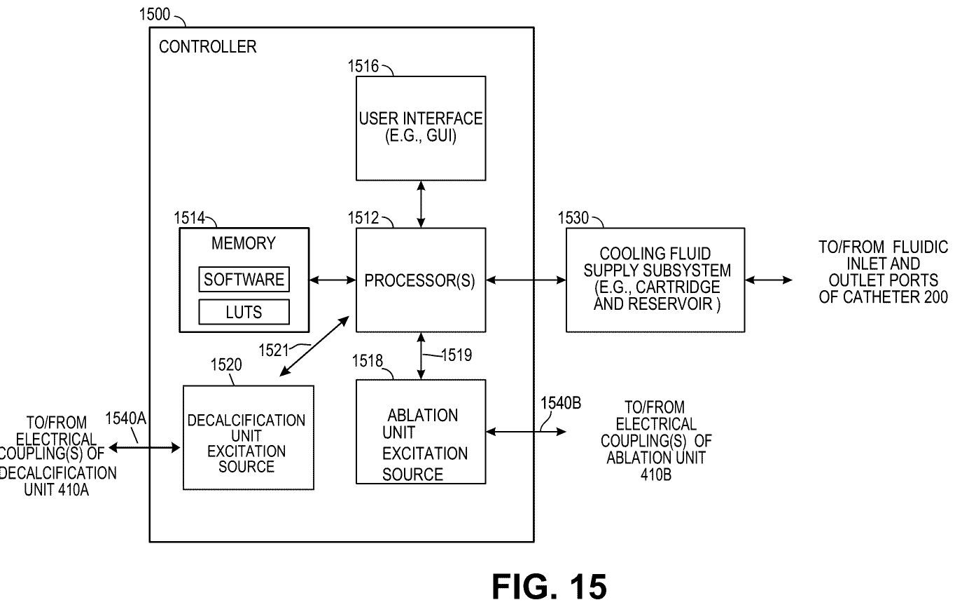

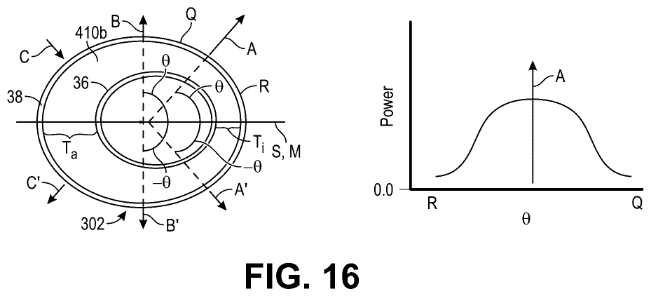

Alternatively or additionally, areas that are free of calcification or less severely affected by calcification may be targeted (provided they are available). For example, an area having a lower calcium score (e.g., a calcium score between 0 to 50) may be favored over an area with a higher calcium score (e.g., a calcium score above 50). In an embodiment, a renal artery may be imaged prior to an ablation procedure to provide a map of calcium scores, i.e., the calcium scores of a plurality of locations along a renal artery. In an embodiment, the renal artery may be imaged, e.g., using an imaging transducer on the treatment catheter 200, close in time, during, and/or after the ablation procedure to provide a map of calcium scores. The map may be provided to the physician/user through a user interface 1516 (FIG. 15).

Calcium scores/calcification scores, as referred to herein, may be qualitatively determined based on an amount of calcification that is visible using an imaging modality. For example, a measurement of calcification may be based on a qualitative determination of whether a location, when viewed using an ultrasound imaging modality, has no plaque, some plaque, a moderate amount of plaque, or a large amount of plaque. Such determinations can be based on relative measurements. For example, if no plaque is visible at a first location, more plaque is visible at a second location, and even more plaque is visible at a third location, then the first location may have a calcium score of 1-10, the second location may have a calcium score of 11-100, and the third location may have a calcium score of 101-400.

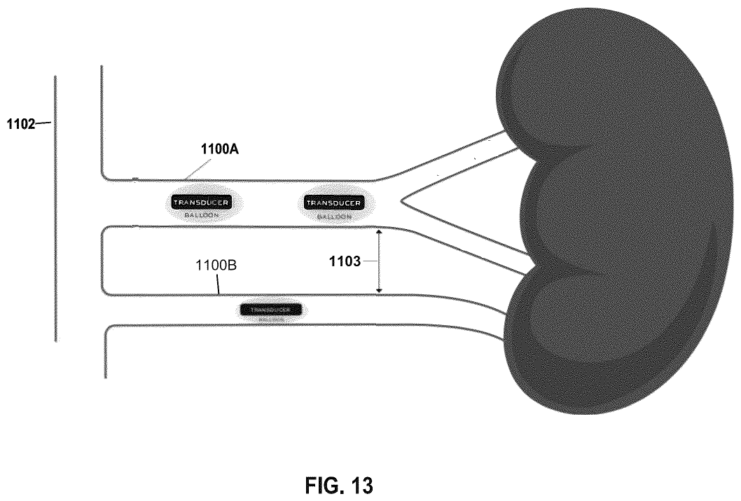

In certain embodiments where an accessory artery, side branch, or vein is uncalcified, less calcified, or has more easily treated calcification (and is therefore a less diseased vessel), and runs parallel with a calcified vessel, nerves running exterior to the calcified vessel may be targeted by sonicating within the less diseased vessel for a longer period to increase the lesion length in order to reach those nerves. As illustrated in FIG. 13, for example, an accessory renal artery 1100b runs parallel with a main renal artery 1100a. If, for example, the calcification score at one or more target locations along a main renal artery 1100a is higher than the calcification score at one or more target locations along the accessory artery 1100b (either after a decalcification/cavitation procedure or in an instance where decalcification is not performed), the ablation procedure may be performed from within the accessory artery 1100b, where the calcification score is lower. If, on the other hand, the calcification score at one or more target locations along a main renal artery 1100a is lower than the calcification score at one or more target locations along the accessory artery 1100b (either after a decalcification/cavitation procedure or in an instance where decalcification is not performed), the ablation procedure may be performed from within the main renal artery 1100a, where the calcification score is lower. An ablation lesion 1103 may extend from about 1 mm exterior to the accessory artery 1100b to about 1 mm exterior to the main renal artery 1100a. In certain embodiments, this may be performed by forming a longer than average ablation lesion between the two vessels. For example, ablation lesions may on average be about 5 mm in length. To extend the ablation lesion from one vessel to another, a 7 mm ablation lesion may be formed. For example, treatment duration (7) may be increased to 10 seconds from 7 seconds, while the flow rate and Acoustic Entry Power are held constant for treatment within a less diseased vessel in order to create a lesion that reaches the exterior wall of a parallel more diseased vessel. In another example, Acoustic Entry Power is increased while treatment duration (7) are held constant for treatment within a less diseased vessel in order to create a lesion that reaches the exterior wall of a parallel more diseased vessel. In another example, Acoustic Entry Power is decreased while treatment duration (7) is increase for treatment within a less diseased vessel in order to create a lesion that reaches the exterior wall of a parallel more diseased vessel, while still compensating for calcification within the less diseased vessel. As illustrated in FIG. 16, ablation transducer unit 401b having multiple focus depths (e.g., due to varying wall thicknesses activated at a variety of optimal frequencies) can be used. In other embodiments, an array of transducers having varying optimized frequencies, may also be used to achieve an ablation lesion reaching the more diseased vessel, while also ablating the less diseased vessel from within the less diseased vessel lumen. While the ablation lesion between the two vessels may be longer than an average ablation depth, the ablation transducer unit 401b may target an average, or close to average, ablation depth in other target areas circumferential to the less diseased vessel/vessel with a lower calcification score.

A transducer having multiple focus depths (e.g., due to varying wall thicknesses activated at a variety of optimal frequencies), or an array of transducers having varying optimized frequencies that may be rotated to focus on a target to ablate nerves running nonparallel, e.g., at an angle, to a more diseased vessel from within the less diseased vessel. As a result of the changing thickness of the ablation transducer unit 401b, the direction that the acoustic signal travels away from the ablation transducer unit 401b changes in response to changes in the frequency of the applied alternating current. The change in direction results from the efficiency of the ablation transducer unit 401b being different for different applied alternating current frequencies. In general, the thickness of the portion of the ablation transducer unit 401b that produces the acoustic signal is inversely proportional to the frequency of the alternating current applied to the ablation transducer unit 401b. As a result, the frequency of the applied alternating current can be tuned so as to tune the portion of the ablation transducer unit 401b that produces the acoustic signal and accordingly tunes the direction that the acoustic signal travels away from the ablation transducer unit 401b. In certain embodiments, a portion of the ablation transducer unit 401b that activates at a lower frequency (a thicker portion) is used to aim at deeper regions (e.g., 4 mm to 10 mm from the lumen of a blood vessel). A portion of the ablation transducer unit 401b that activates at a higher frequency (a thinner portion) is used to target nearer regions (e.g., 0.5 mm to 4 mm from the lumen of a blood vessel).

FIG. 16 has an arrow labeled A that represents a direction that an acoustic signal travels away from the ablation transducer unit 401b. The direction that the acoustic signal travels away from the ablation transducer unit 401b can be represented by an angle labeled e, where e is measured relative to a measurement line such as the measurement line labeled S in FIG. 16. Examples of suitable measurement lines include, but are not limited to, a line of symmetry for the outer surface of the transducer. In some instances, the measurement line extends through the center of the outer surface of the transducer, the center of gravity of the outer surface of the transducer and/or the center of gravity of the transducer.

FIG. 16 also includes a graph of an exemplary power distribution for the acoustic signal labeled A. The power distribution shows the power level of the acoustic signal labeled A from the angle θ labeled R to the angle θ labeled Q. The angle θ associated with the acoustic signal labeled A (the representative angle) can be chosen to be representative of the direction that the acoustic signal labeled A travels away from the transducer. For instance, the representative angle associated with the acoustic signal labeled A can be located at the maximum in the power distribution, the average of the power distribution over a range of angles θ, the average of the power distribution weighted by power and taken over a range of angles 30°, 90°, or 180°.

In order to illustrate the steerable nature of the acoustic signal, FIG. 16 also includes arrows labeled B and C. Each of the arrows labeled A, B, and C represents a direction that a different acoustic signal travels away from the ablation transducer unit 401b. The acoustic signal represented by the arrow labeled A occurs when a higher alternative current frequency (“a first frequency”) is applied to the ablation transducer unit 401b than the acoustic signal represented by the arrow labeled B. Additionally, the acoustic signal represented by the arrow labeled B occurs when a higher alternative current frequency (“a second frequency”) is applied to the ablation transducer unit 401b than the acoustic signal represented by the arrow labeled C. And the acoustic signal represented by the arrow labeled C occurs when a lower alternative current frequency (“a third frequency”) is applied to the ablation transducer unit 401b than the acoustic signal represented by the arrows labeled B or A. Accordingly, different angles are associated with different alternating current frequency levels.

The measurement line labeled S in FIG. 16 is a line of symmetry. In an embodiment, a first region of the ablation transducer unit 401b on one side of the line of symmetry has a thickness that is the same as a second region of the transducer on the opposite side of the line of symmetry. As a result, the ablation transducer unit 401b can concurrently output the same acoustic signals from opposite regions of the ablation transducer unit 401b. For instance, the transducer of FIG. 16 can concurrently output an acoustic signal labeled A and an acoustic signal labeled A′ equal in power at the first frequency, or can concurrently output the acoustic signal labeled B and an acoustic signal labeled B′ equal in power at the second frequency, or can concurrently output the acoustic signal labeled C and an acoustic signal labeled C′ equal in power at the third frequency. As a result, a single alternating current frequency level can be associated with more than one angle θ.

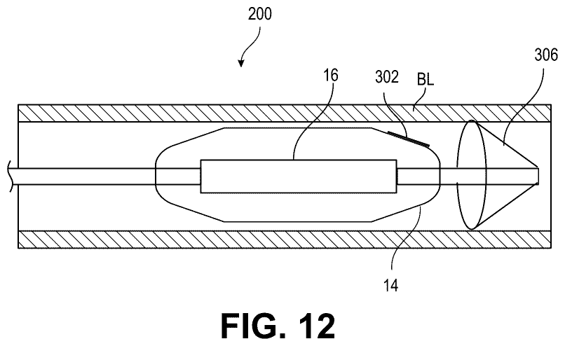

The ablation transducer unit 401b of FIG. 16 can be modified such that when the ablation transducer unit 401b has multiple different regions of the same thickness, the region from which the acoustic signal is output can be selected. U.S. application Ser. No. 18/451,044, filed Aug. 16, 2023, FIGS. 12A-17 and paragraphs [0167]-[0226], incorporated herein by reference, disclose further ablation transducer units 401b that can target tissue in specific directions.

Referring to FIG. 3, as the length of a transducer 16 becomes smaller, the number of lobes, corresponding to output from the transducer 16, can also decrease. For example, a transducer 16, comprising a length in a range of approximately 2.5 mm to approximately 3 mm and operating at a frequency of approximately 9 MHz, may produce output, corresponding to a single lobe. In certain embodiments, each of the transducers 16 is optimized for ablation, as described herein, and transducer cavitation unit 401a is substituted with a second transducer ablation unit 401b. In this example, one or more electrode cavitation units on the expandable member 14 are disposable between the lobes corresponding to output from different transducers 16. For instance, one or more electrode cavitation units on the expandable member 14 are disposable over components of the catheter 12 located between adjacent transducers 16.

The device 200 optionally comprises at least one flexible feature 402 such as bridge portions 113 having enhanced flexibility regions that do not extend under the transducers 16. Alternatively, each bridge portion 113, disposed in the catheter 12, comprises at least one enhanced flexibility region L s extending between two transducers 16 and under at least one transducer 16.

Still referring to FIG. 3, together, a fluid lumen 40 is disposed in relation to the catheter shaft 12a. The lumen 40 comprises a fluid port (not shown) through which the fluid lumen 40 and the interior of the expandable member 14 can exchange fluid. As a result, the fluid lumen 40 and the interior of the expandable member 14 are in fluid communication. Accordingly, the fluid lumen 40 provides fluid communication between an interior of the expandable member 14 and one of several conduits (not shown). The catheter system 100 (FIG. 1) can be configured to drive fluid through the fluid lumen 40 into the interior of the expandable member 14 and/or to withdraw fluid from the interior of the expandable member 14 through the fluid lumen 40. As a result, the fluid can be used to inflate or deflate the expandable member 14. Alternatively, the catheter shaft 12a comprises several fluid lumens 40 that are each open to an interior of the expandable member 14. The catheter system 100 can be configured to drive fluid through a first selection of the fluid lumens 40 into the interior of the expandable member 14 and to withdraw fluid from the interior of the expandable member 14 through a second selection of the fluid lumens 40. The relative flow of fluid into the expandable member 14 and from the expandable member 14 can be varied so as to inflate the expandable member 14, deflate the expandable member 14, or keep the expandable member 14 inflation level at steady state.

Still referring to FIG. 3, together, the catheter shaft 12a comprises an electrical lumen (not shown). The electrical lumen comprises a first conductor carrier (not shown) and a second conductor carrier The first conductor carrier extends through a wall of the catheter shaft 12a into the interior of the expandable member 14. The first conductor carrier includes a first electrical conductor (not shown) configured to couple with the backing member 42. An electrically conducting backing member 42 and an electrically conducting spacing component provide electrical communication between the inner electrode 36 and the first electrical conductor. Additionally, the first electrical conductor is in electrical communication with the electronics (not shown) through an electrical coupling (not shown) and one of the external electrical conductors.

Still referring to FIG. 3 together, at least a portion of bridge portion 113 of the backing member 42 extending between adjacent transducers 16 can include one or more enhanced flexibility regions 114. The one or more enhanced flexibility regions 114 can be selected to enhance the flexibility of the backing member 42 and accordingly the catheter 12. Suitable enhanced flexibility regions 114 include, but are not limited to, openings through the wall of the backing member 42 arranged in patterns, e.g., the backing member 42 having lattice patterns.

Still referring to FIG. 3, together, the enhanced flexibility region 114 comprises an opening 116 through the wall of the backing member 42. The opening 116 spirals around a longitudinal axis of the backing member 42 for the portion of the backing member 42 that is located between adjacent transducer assemblies 32. As a result, at least one enhanced flexibility region 114 of the backing member 42 has a helical, or substantially helical, configuration for a portion of the longitudinal length of the backing member 42. In some instances, the enhanced flexibility region 114 does not extend into any of the transducer assemblies 32 in the expandable member 14.

Still referring to FIG. 3, together, the spiral rate can measure the number of degrees that the helix turns around the longitudinal axis of the backing member 42 per length of the longitudinal axis. The spiral rate can determine the degree of flexibility of the enhanced flexibility region 114 of the backing member 42. For instance, increasing the spiral rate can provide more flexibility to the backing member 42 while decreasing the spiral rate can provide more rigidity to the backing member 42. Suitable spiral rates (pitch counts) include, but are not limited to, rates greater than or equal to approximately 0°/mm and can extend over an angular range of more than approximately 360° or an angular range of more than approximately 720°.

Still referring to FIG. 3, together, a connecting portion 96 of the backing member 42 extends from a transducer 16, to at least a portion of the catheter shaft 12a of the catheter 12. The connecting portions 96 are shown as excluding one or more enhanced flexibility regions 114; however, the connecting portions 96 optionally comprise one or more enhanced flexibility regions 114. Although the catheter device 200 is shown as comprising two units 401a,b disposed in a single expandable member 14, such as a balloon, the expandable member 14 can accommodate more than two units 401, e.g., respectively comprising transducers 16.

Still referring to FIG. 3, when the catheter device 200 comprises at least three units 401, e.g., respectively comprising three transducers 16, the backing member 42 comprises several bridge portions 113. At least one bridge portion 113 of the plurality of bridge portions 113 comprises at least one enhanced flexibility region 114. In some instances, the number of transducer assemblies 32, e.g., respectively comprising transducers 16, disposed in the expandable member 14, comprises a range of approximately 2 to approximately 20. In an example suitable for use in treating a renal artery, by example only, the number of units 401, e.g., respectively comprising transducers 16 disposed in the expandable member 14 comprises a range of approximately 2 to approximately 5.

Still referring to FIG. 3, although electrodes are not shown as disposed on the expandable member 14, the expandable members 14 are also configured to optionally accommodate at least two electrodes. When the expandable member 14 optionally accommodates the at least two electrodes, a second conductor carrier provides electrical communication between any two electrodes and the external electronics. When the expandable member 14 optionally accommodates the at least two electrodes, at least a portion of the transducers 16 is associated with a different selection of the at least two electrodes.

Additionally, the external electronics can independently activate selection of each at least two electrodes. As a result, the external electronics can deliver ultrasonic energy from a selection of the expandable members 14 and can deliver electromagnetic energy from the at least two electrodes associated with at least two portions of the expandable members 14 in the selection of the expandable members 14.

The cavitation transducer unit 401a and ablation transducer unit 401b may be configured in many shapes, such as a cylindrical shape, e.g., a tube, or a flat shape, e.g., a flat rectangular or a disc shape.

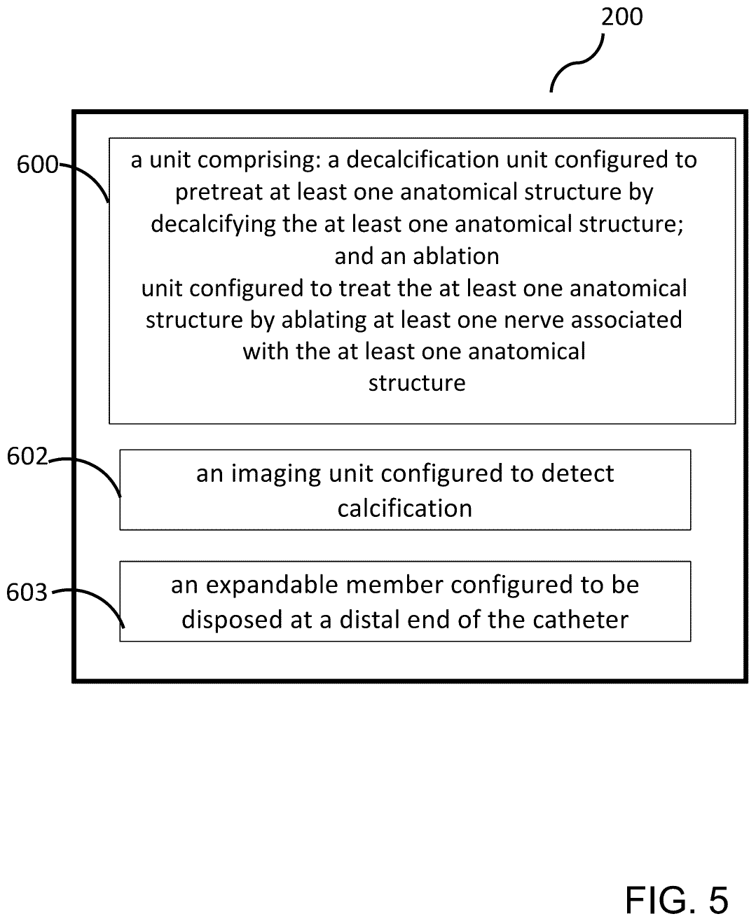

Referring to FIGS. 4A-5, together, these schematic diagrams illustrate various embodiments of a catheter device 200 for pretreating and treating an anatomical structure, in accordance with an embodiment of the present disclosure. The catheter device 200 comprises, with reference to FIG. 5, several units 600. As shown in FIGS. 4A-4D, the plurality of units may comprise one or more ablation and/or decalcification transducers 500, one or more decalcification and/or ablation electrodes 510, and one or more imaging transducers 520. In certain embodiments, catheter 200 can be configured for chemical ablation. In certain embodiments, catheter 200 can be configured to ablate renal fibers innervating a kidney using direct current heating, cryogenics, intravascular or extracorporeal focused ultrasound, microwave heating, laser heating, induction heating, radiation, or mechanical methods. As shown in the schematics of FIGS. 4A-4D, the electrodes 510 and one or more imaging transducers 520 may be interspersed among the transducers 500 along the catheter shaft 12a in various non-limiting configurations. In an embodiment, an imaging transducer 520 having a frequency in the range of 30 to 45 MHz is used to provide high-resolution intravascular images.

In some embodiments, a catheter 200 comprises a plurality of units 600: comprising a decalcification unit configured to pretreat the at least one anatomical structure by at least partially decalcifying the at least one anatomical structure; and an ablation unit configured to treat the at least one anatomical structure by ablating at least one nerve associated with the at least one anatomical structure. At least one unit of the plurality of units can be configured to be disposed in a catheter 12. The length of the decalcification transducer or ablation transducer 500 depends on various factors such as the site of treatment, etc. However, in some embodiments, the length of a cylindrical decalcification or ablation transducer 500 may be between 0.9 millimeter (mm) to 30 mm. In some embodiments, the length of each cylindrical decalcification or ablation transducer 500 may be between 1 to 10 mm. In some embodiments, the length of each cylindrical decalcification or ablation transducer 500 may be between 5 to 6 mm. In some embodiments, the length of each decalcification or ablation transducer 500 may be between 0.5 to 2 mm.

In certain embodiments described herein, the decalcification transducer or the ablation transducer 500 may have an outer diameter of about 1.3 mm and an operating frequency of 11 to 15 MHz. The ultrasound transducer 500 can be configured to deliver sufficient acoustic energy during sonication such as to thermally induce modulation of neural fibers surrounding a blood vessel sufficient to improve a measurable physiological parameter corresponding to a diagnosed condition of the patient. In an embodiment, the generator may be configured to energize the decalcification transducer or ablation transducer 500, for a time period of between 5 to 20 seconds, at a frequency of 11 to 15 MHz, or both. In an embodiment, the generator may be configured to energize the decalcification transducer or ablation transducer 500 for a time period of between 6 to 10 seconds, at a frequency of 12 to 14 MHz, or both. In an embodiment, the generator may be configured to energize the decalcification transducer or ablation transducer 500 for a time period of about 7 seconds, at a frequency of about 13 MHz, or both. Energizing the decalcification transducer or ablation transducer 500 by the generator may increase a temperature of the decalcification transducer or ablation transducer 500 by no more than 50° C. Energizing the decalcification transducer or ablation transducer 500 by the generator delivers energy at an average surface acoustic intensity of between 20 and 150 W/cm². In an embodiment, the decalcification transducer or the ablation transducer 500, may be energized for a time period of between 5 to 20 seconds, at a frequency of 11 to 15 MHz, or both. In an embodiment, the piezoelectric component may be energized for a time period of between 6 to 10 seconds, at a frequency of 12 to 14 MHz, or both. In an embodiment, the piezoelectric component may be energized for a time period of about 7 seconds, at a frequency of about 13 MHz, or both. In an embodiment, energizing the piezoelectric component may increase a temperature of the piezoelectric component by no more than 50° C. In an embodiment, energizing the piezoelectric component may deliver energy at an average surface acoustic intensity of between 20 and 150 W/cm². At 15 MHz, more of the energy delivered is delivered at a distance within 6 mm or less (65% versus 46% for 9 MHz), improving energy targeting specificity and safety. Comparing the heating power profiles of the decalcification transducer or ablation transducer 500 operating at 12 MHz versus 15 MHz, the 12 MHz transducer has greater heating power at distances of less than about 6 mm and lesser heating power at distances of greater than about 6 mm.

The decalcification transducer or the ablation transducer 500 may be configured to resonate at a frequency between 200 kHz to 1 MHz. Optionally, the decalcification transducer or the ablation transducer 500 may be configured to resonate at a frequency between 500 kHZ to 1 MHz. Alternatively, the decalcification transducer or the ablation transducer 500 may be configured to resonate at a frequency between 600 kHz to 900 kHz. In some embodiments, the ablation transducer 500 may be activated at an intensity between 10.0 to 90.0 Watts per cm² for a time between 5 to 20 seconds, and at a frequency of between 8.5 MHz to 15 MHz.

In certain embodiments, decalcification is not performed and/or the calcification is only partially fragmented. The calcification may be otherwise compensated for, as provided herein according to certain embodiments.

Referring to FIG. 4A, this diagram illustrates, in a side view, one embodiment of a catheter device 200 for pretreating and treating an anatomical structure, such as a blood vessel, in accordance with an embodiment of the present disclosure. For example, the catheter device 200 comprises a transducer 500 disposed around a catheter shaft 12a and two ablation electrodes 510 disposed around the catheter shaft 12a and are respectively disposed forward and aft of the transducer 500.

Referring to FIG. 4B, this diagram illustrates, in a side view, another embodiment of a catheter device for pretreating and treating an anatomical structure, in accordance with an embodiment of the present disclosure. For example, the catheter device 200 comprises two transducers 500 disposed around a catheter shaft 12a and three ablation electrodes 510 disposed around the catheter shaft 12a and separating the transducers 500. More particularly, an intermediate ablation electrode 510 can be longitudinally between the transducers 500, and a proximal ablation electrode and distal ablation electrode 510 can bookend (be disposed forward and aft of) the transducers 500.

Referring to FIG. 4C, this diagram illustrates, in a side view, another embodiment of a catheter device for pretreating and treating an anatomical structure, in accordance with an embodiment of the present disclosure. For example, the catheter device 200 comprises: a transducer 500 disposed around a catheter shaft 12a, two ablation electrodes 510 disposed around the catheter shaft 12a and are respectively disposed forward and aft of the transducer 500; and a step 521 for connecting the transducer 500 and one ablation electrode 510 of the two ablation electrodes 510.

Referring to FIG. 4D, this diagram illustrates, in a side view, another embodiment of a catheter device for pretreating and treating an anatomical structure, in accordance with an embodiment of the present disclosure. For example, the catheter device 200 comprises two transducers 500 disposed around a catheter shaft 12a, three ablation electrodes 510 disposed around the catheter shaft 12a and separating the transducers 500, and an imaging transducer 520 disposed around the catheter shaft 12a and between one transducer 500 of the two transducers 500 and one ablation electrode 510 of the two ablation electrodes 510.

Delivering bubbles adjacent to a calcified region or at the calcified region may depend on the distance between the expandable member 14, e.g., a balloon and the internal surface of the blood vessel, e.g., renal artery. A smaller distance between the balloon and the internal surface of the renal artery may increase the chances that cavitation bubbles will form adjacent and/or at the internal surface of the renal artery. In other words, once the deflated balloon is advanced and placed adjacent to a calcified region or at the calcified region, the balloon is expanded. The fluid in the balloon may act as a coupler to facilitate efficient energy transfer of the pressure waves from the decalcification electrodes 510 into the surface of the blood vessel to reach the calcified region.

Referring to FIG. 5, in the system 100, the decalcification unit comprises at least one of a decalcification transducer 500 and/or several decalcification electrodes 510. In some embodiments, the decalcification transducer is configured to effect decalcification in a frequency range of approximately 400 kHz to approximately 3 MHz. The plurality of decalcification electrodes are configured to generate bubbles in a liquid and cavitation, whereby cavitation is affected, and whereby calcification is decoupled from the at least one anatomical structure such that the at least one anatomical structure is substantially unharmed. The plurality of decalcification electrodes can be configured in an array. The plurality of decalcification electrodes comprises at least one ring electrode. At least one electrode of the plurality of decalcification electrodes is configured to be disposed in at least one of: a distal end of the catheter, proximal at least one unit of the plurality of units 600, on at least one unit of the plurality of units 600.

Still referring to FIG. 5, in the system 100, the ablation unit comprises at least one of an ablation transducer 500 or several of ablation electrodes 510. In some embodiments, the ablation transducer is configured to effect denervation at a frequency of approximately 9 MHz. The plurality of ablation electrodes comprises at least one radio-frequency electrode. The at least one radio-frequency electrode is configured to perform at least one of ablation, confirmation, or mapping of at least one nerve.

Still referring to FIG. 5, in some embodiments of the system, 100, either the plurality of units 600 or the ablation unit further comprises an imaging unit 602. The imaging unit 602 may comprise an imaging transducer (not shown) configured to identify calcification and/or the media-adventitia border. The imaging transducer is configured to image at a frequency of approximately 20 MHz-60 MHz. In some embodiments, the imaging transducer comprises a piezoelectric material. Suitable materials for the imaging transducer include, but are not limited to, piezoelectric materials including piezoelectric ceramics, crystalline and polymers, acoustic micro-electro mechanical systems (MEMS) transducers such as piezoelectric micromachined ultrasonic transducers (PMUT) and capacitive micromachined ultrasonic transducer (CMUT). Examples of suitable piezoelectric include, but are not limited to, lead zirconate titanate (PZT), CMUT, and PMUT. The imaging transducer can comprise elements that are rectangular. The element size is determined by the field of view. The elements of the transducer can be configured in an array, or the transducer can have a single element. The element can be located on a step of the transducer 500 or the element can be located separately from the transducer 500. The array element can have a dimension of 0.3 millimeter to 1.5 millimeter in height and 0.5 wavelength to 2 wavelength in width. The array can be a single cylindrical array or a multi-row cylindrical array. The multi-row cylindrical array helps reduce the image slice thickness to achieve better contrast resolution. To reduce the number of cables, the elements can be individually controlled to transmit and receive by an ASIC circuit. The array size can be 8 to 256 elements. The imaging unit 403 can be located between the tip member 18 and the expandable member 14 or it can be located on the proximal end of the expandable member 14. The imaging depth can be up to 12 millimeters. The imaging unit 403 can be used to size the vessel, image the anatomy, pathology including the vessel wall, plaques, calcification, tissue layers and nerves.

Still referring to FIG. 5, the catheter device 200 further comprises at least one of an expandable member 603 and a porous member (not shown). At least one of the expandable member 603 and the porous member is configured to be disposed at a distal end of the catheter 12, e.g., at a distal end 22 of a catheter shaft 12a (FIG. 1). The expandable member 603 comprises at least one material of: a polyamide, a polyimide film, a polyethylene terephthalate, a thermoplastic elastomer, a nylon, a PEBAX™ thermoplastic elastomer, a medical-grade thermoplastic polyurethane elastomer, a PELLETHANE™ thermoplastic polyurethane elastomer, a pellethane, an isothane, and any other suitable polymer or any combination thereof.

In certain embodiments, the catheter device 200 further comprises a flexible feature 402 (FIG. 3A) configured to separate each unit 600 from another unit 600 of the plurality of units 600, at least a portion of the flexible feature 402 configured to be disposed in the catheter 12. The flexible feature 402 is configured to facilitate navigating the catheter 12 through the at least one anatomical structure. The distance between any of the transducers 500 and decalcification electrodes 510 may assist in catheter flexibility due to the gaps separating them from each other.

Still referring to FIG. 5, in the catheter device 200, each unit of the plurality of units 600 is configured to selectively operate in at least one mode of an independent mode (e.g., independent of a mode of any other of the one or more units) and a dependent mode (e.g., its function is dependent on a mode of another unit). Each unit of the plurality of units 600 is configured to selectively operate in at least one ultrasound mode of a focused mode and an unfocused mode. In some embodiments, the first decalcification electrode 510 and the second decalcification electrode 510 may be connected in series such that activating the first decalcification electrode 510 also activates the second decalcification electrode 510. This may allow the decalcification electrodes 510 to generate up to two shock waves using one generator.

Still referring to FIG. 5, in the catheter device 200, the plurality of decalcification electrodes comprises at least one shockwave electrode. The plurality of decalcification electrodes comprises at least one material of metal, such as stainless steel, tungsten, nickel, iron, and steel. The catheter device 200 is suitable for use with the at least one anatomical structure comprising at least one vasculature, the at least one vasculature comprising at least one of a renal vein and a renal artery.

Still referring to FIG. 5, the catheter device 200 is provided and is configured to pretreat renal arteries by at least partially removing calcium deposits from the arterial wall and/or ablating nerves to treat a medical condition, e.g., hypertension, arrhythmia, heart failure, chronic kidney disease, atrial fibrillation, end stage renal disease, myocardial infarction, anxiety, diabetes, metabolic disorder and insulin resistance. Once the catheter device 200 is positioned near the calcified region, the user can start with low energy shock waves and increase the energy as needed to at least partially fragment calcification of the calcified region. The shock waves can be conducted through fluid, through the expandable member 14, through the blood and vessel wall to the calcified region where the energy can break/fragment the calcified region without the application of excessive pressure by the expandable member 14 on the walls of the blood vessel. In some embodiments, the catheter device 200 is further configured to identify calcified plaque, prior to applying an ablation therapy, and to verify that the plaque has been removed, e.g., after applying an ablation therapy. For example, such calcified plaque identification and/or plaque removal verification may be performed and/or confirmed by using an imaging unit/device. Intravascular imaging may be performed either with intravascular ultrasound or optical coherence tomography in order to define the calcium density and determine the cavitation parameters. In an embodiment, the imaging unit/device may be the same imaging unit/device used for identifying the calcified region and for determining whether calcification has been at least partially fragmented from the calcified region. In another embodiment, there may be a separate imaging unit/device for identifying a calcified region and a separate imaging unit/device for determining whether calcification has been at least partially fragmented from the calcified region.

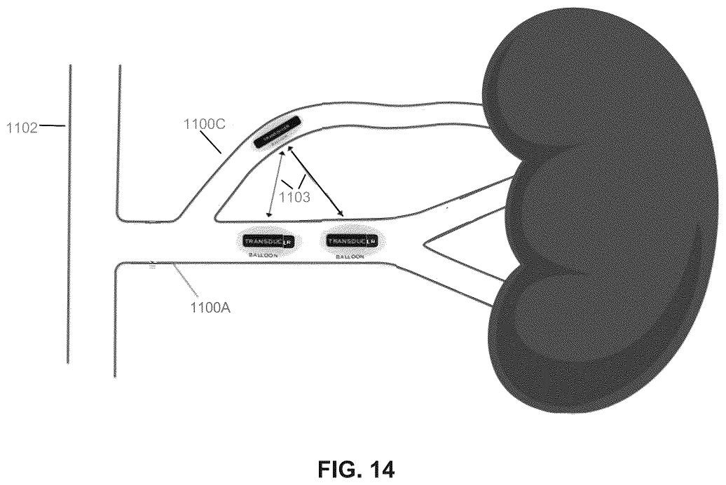

In certain embodiments, a high-resolution intravascular ultrasound imaging transducer is used to detect the media-adventitia border. One or more processors 1512 may determine that the media-adventitia border is not detectible using the intravascular ultrasound imaging, either because a user enters this determination into user interface 1516 or because the one or more processors 1512 are configured to determine that an image input into the controller 1500 does not include a media-adventitia border, e.g., using a machine learning model implemented by at least one of the one or more processors, or more generally, using artificial intelligence. If processor 1512 determines that the media-adventitia border is not detectible, the processor 1512 may determine that cavitation/decalcification is necessary or alternative treatment parameters (e.g., reduced power, longer duration, and/or increased flow rate) are warranted and/or an alternative treatment location is advisable. If the media-adventitia border is detectible using the imaging transducer than the processor may determine that the cavitation operation may be skipped and ablative treatment commenced and/or the processor may save this location as a candidate for treatment. A user may also input a calcium score at a given location based on the image provided by the intravascular ultrasound imaging transducer or the processor may be configured to determine a calcium score directly from inputted images. A calcified region/plaque should have a calcium score in the range of low to intermediate. A low calcium score is in the range of 0-50 and an intermediate calcium score is in the range of 50-1000. When the calcium score is lower, decalcification may be more effective and/or more easily compensated for by changing the treatment parameters (e.g., reduced power, longer duration, and/or increased flow rate), thus improving the efficacy of ablation of the one or more nerves within and/or surrounding the decalcified region of the vessel. If the calcium score is too high, the processor 1512 may determine that treatment at this location should be avoided or alternative treatment strategies (e.g., ablation of adjacent nerves from other location as show in FIGS. 13 and 14) should be performed.

In certain embodiments, the imaging operation is omitted, and a cavitation procedure is performed regardless of a location's calcium score.

In an embodiment, impedance (e.g., blood impedance) is used to determine calcification without the need of an imaging device. If the impedance indicates calcification, the area is cavitated and impedance is tested again to determine whether the target location has been sufficiently decalcified.

Still referring to FIG. 5, the system 100, in some embodiments, is configured to operate as an ultrasound renal-denervation (uRDN) system. In some embodiments, the catheter device 200 comprises: a first transducer unit 500, such as an ablation transducer unit, optimized for denervation therapy. In some embodiments, as shown in FIG. 4A, the ablation transducer unit comprises an ablation transducer, e.g., an unfocused transducer, operable at a frequency of approximately 9 MHz or in a range of approximately 6 MHz to approximately 20 MHz. In some embodiments, as shown in FIG. 4C, the catheter device 200 further comprises a second transducer unit 520, such as an imaging transducer unit, optimized for imaging. In an embodiment comprising an imaging transducer unit, the imaging transducer unit comprises an imaging transducer comprising a piezoelectric material. The piezoelectric material may have an operable frequency, e.g., of approximately 40 MHz. The operable frequency can be configured, e.g., by material selection, to identify a calcium deposit.

In some embodiments, as shown in FIG. 4D, the catheter device 200 comprises a third transducer unit 530, such as a decalcification transducer unit optimized for breaking/fragmenting calcification. The decalcification transducer unit comprises a decalcification transducer, e.g., a focused transducer, having an operable frequency in a range of approximately 400 kHz to approximately 3 MHz. The operable frequency of the decalcification transducer is configured to introduce cavitation and to create micro-cracks within the calcified plaques without substantially damaging the arterial wall.

Referring to FIGS. 4A-4D, in some embodiments, instead of, or in addition to, a third transducer unit 530, the catheter device 200 comprises multiple electrodes. For example, at least one array of electrodes 510, such as decalcification electrodes, configured to generate vapor bubbles and subsequent cavitation to crack calcification from the arterial wall without substantially damaging the arterial wall. The decalcification electrodes comprise at least one ring electrode coupled with a catheter, e.g., distal and/or proximal the transducer units and within an insulated balloon to protect a patient from an electric shock. In an embodiment, the ring electrodes are located on the transducer itself, e.g., on one or more operations or setbacks of the transducer, in order to minimize the space taken by the ring electrodes. In another embodiment, one ring electrode could be disposed distal or proximal a transducer unit 500 and another ring electrode could be disposed between two transducer units 500. In one embodiment, as shown in FIG. 4B, the catheter device 200 comprises two transducer units 500 optimized for denervation therapy; and the ring electrodes 510 may be distributed in various configurations around the transducer units. In an alternative embodiment, the catheter device 200 comprises a telescoping transducer that slides over the ring electrodes after applying the shock therapy.

Referring back to FIG. 5, in an embodiment, the catheter device 200 comprises a set of electrodes, e.g., attached to the balloon, for treatment confirmation/nerve mapping, such as nerve-mapping electrodes. The electrodes could also be located elsewhere and may comprise ultrasound transparent material or be located on the catheter such that they do not interfere with sonication.

Still referring to FIG. 5, in an embodiment, RF ablation electrodes are provided instead of an ablation transducer. The RF ablation electrodes are operable to perform treatment confirmation/nerve mapping. The RF ablation electrodes are disposable proximal and/or distal the insulated balloon and/or attached to the balloon surface. In an embodiment, a distal balloon or basket impedes emboli from traveling within distal branches of the renal artery and/or the kidney. The trapped embolism can then be removed by aspiration towards the proximal end.

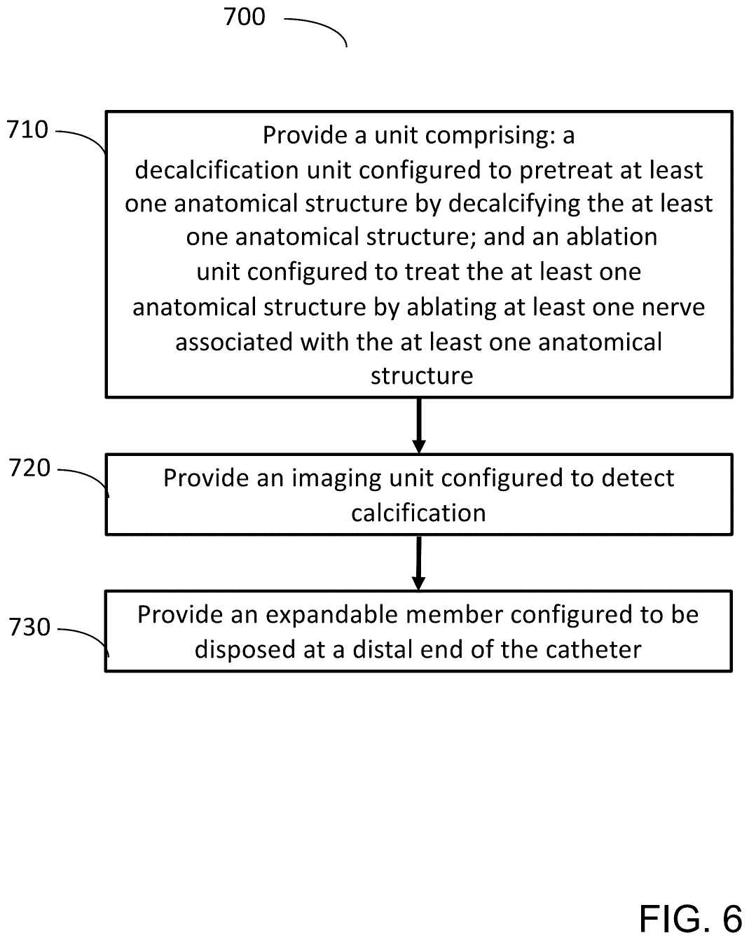

Referring to FIG. 6, this flow diagram illustrates a method 700 of fabricating a catheter device 200 for pretreating and treating at least one anatomical structure, in accordance with an embodiment of the present disclosure. The method 700 comprises: providing several units 600. Providing the plurality of units 600 comprises: providing a decalcification unit configured to pretreat the at least one anatomical structure by decalcifying the at least one anatomical structure; and providing an ablation unit configured to treat the at least one anatomical structure by ablating at least one nerve associated with the at least one anatomical structure, as indicated by block 710; and providing an imaging unit configured to detect calcification, as indicated by block 720. At least one unit of the plurality of units is configured to be disposed in a catheter.

Still referring to FIG. 6, in the method 700, providing the decalcification unit (block 710) comprises providing at least one of a decalcification transducer and several decalcification electrodes. The method 700 further comprises configuring the decalcification transducer to effect decalcification in a frequency range of approximately 400 kHz to approximately 3 MHz. The method 700 further comprises configuring the plurality of decalcification electrodes to generate bubbles in a liquid and cavitation, whereby cavitation is effected, and whereby calcification decouples from the at least one anatomical structure such that the at least one anatomical structure is substantially unharmed. The method 700 further comprises configuring the plurality of decalcification electrodes in an array. In some embodiments, providing the plurality of decalcification electrodes comprises providing at least one ring electrode.

Still referring to FIG. 6, the method 700 further comprises configuring at least one electrode of the plurality of decalcification electrodes to be disposed in at least one of: a distal end of the catheter, proximal at least one unit of the plurality of units 600, on at least one unit of the of the plurality of units 600, on at least one step (or setback) of at least one unit of the plurality of units 600, between two units of the plurality of units 600, proximal an expanding member, and a location that does not interfere with sonication.

Still referring to FIG. 6, in the method 700, providing the ablation unit comprises providing at least one of an ablation transducer and several ablation electrodes. The method 700 further comprises configuring the ablation transducer to effect denervation at a frequency of approximately 13 MHz. The method 700 further comprises configuring the ablation transducer to effect denervation in a frequency range of approximately 9 MHz to approximately 15 MHz. Providing the plurality of ablation electrodes comprises providing at least one radio-frequency electrode. The method 700 further comprises configuring the at least one radio-frequency electrode to perform at least one of ablation (of calcification), confirmation (of ablation), and mapping of at least one nerve.

Still referring to FIG. 6, in the method 700, providing one of the plurality of units 600 and the ablation unit further comprises providing an imaging unit 602, as indicated by block 720. Providing the imaging unit 602 comprises providing an imaging transducer configured to identify calcification. The method 700 further comprises configuring the imaging transducer to image at an optimal frequency of approximately 40 MHz, with a range frequency of approximately 20 MHz to approximately 60 MHz, or approximately 30 MHz to 45 MHz. In some embodiments, providing the imaging transducer comprises providing a piezoelectric material.

Still referring to FIG. 6, the method 700 further comprises providing at least one of an expandable member 603 and a porous member (not shown), as indicated by block 730. The method 700 further comprises configuring at least one of the expandable member 603 and the porous member to be disposed at a distal end of the catheter 12. The method 700 further comprises configuring the expandable member 603 to impede at least one embolism, whereby the at least one embolism is removable from the at least one anatomical structure. The method 700 further comprises configuring the porous member to capture at least one embolism, whereby the at least one embolism is removable from the at least one anatomical structure. In some embodiments, a porous member may comprise a stentriever, a basket, a net, or the like. Alternatively, or additionally, aspiration may be used to capture the at least one embolism.

Still referring to FIG. 6, in the method 700, providing the expandable member 603 comprises providing at least one material of: a polyamide, a polyimide film, a polyethylene terephthalate, a thermoplastic elastomer, a nylon, a PEBAX™ thermoplastic elastomer, a medical-grade thermoplastic polyurethane elastomer, a PELLETHANE™ thermoplastic polyurethane elastomer, a pellethane, an isothane, and any other suitable polymer or any combination thereof.

Still referring to FIG. 6, the method 700 further comprises providing a flexible feature 402 (FIGS. 3A-3C) configured to separate each unit from another unit of the plurality of units 600, at least a portion of the flexible feature 402 configured to be disposed in the catheter 12. The flexible feature 402 is configured to facilitate navigating the catheter 12 through at least one anatomical structure.

Still referring to FIG. 6, the method 700 further comprises configuring each unit of the plurality of units to selectively operate in at least one mode of an independent mode (independent of a mode of any other of the one or more units) and a dependent mode (its function is dependent on a mode of another unit). The method 700 further comprises configuring each unit of the plurality of units 600 to selectively operate in at least one ultrasound mode of a focused mode and an unfocused mode.

Still referring to FIG. 6, in the method 700, providing the plurality of decalcification electrodes comprises providing at least one shock wave electrode. Providing the plurality of decalcification electrodes comprises providing electrodes formed of at least one material of: stainless steel, tungsten, nickel, iron, and steel. In the method 700, the at least one anatomical structure comprises at least one vasculature; and the at least one vasculature comprises at least one of: a renal vein and a renal artery.

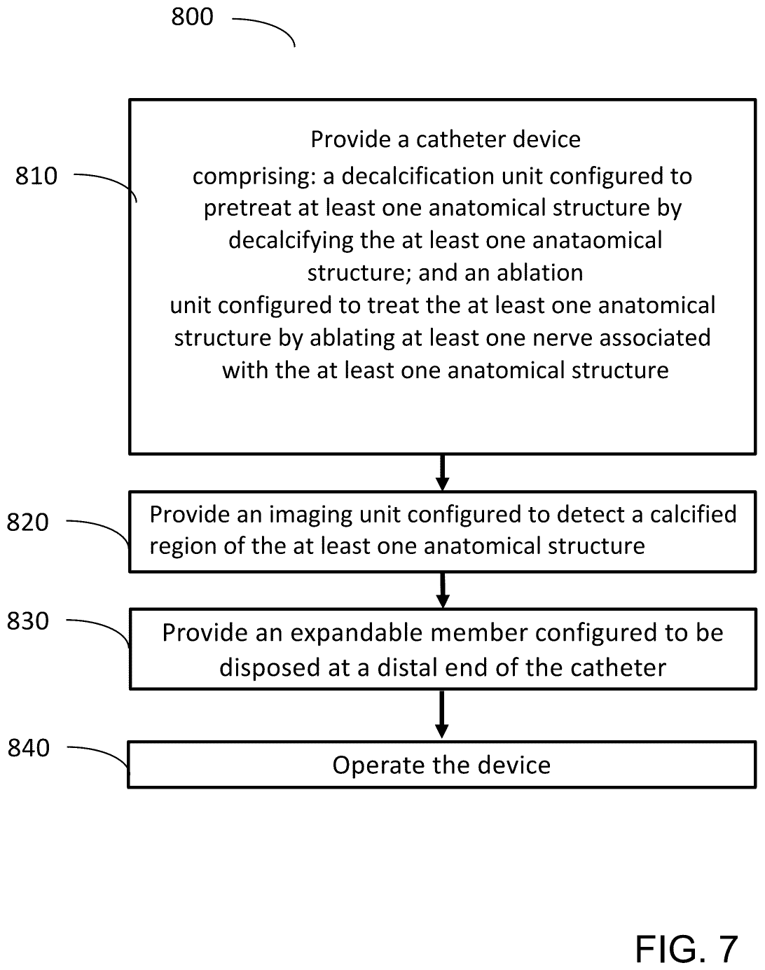

Referring to FIG. 7, this flow diagram illustrates an embodiment of a method 800 of pretreating and treating at least one anatomical structure by way of a catheter device 200, in accordance with an embodiment of the present disclosure. The method 800 comprises: providing the catheter device 200, as indicated by block 810, comprising a unit 600, comprising: a decalcification unit configured to pretreat the at least one anatomical structure by decalcifying the at least one anatomical structure; and an ablation unit configured to treat the at least one anatomical structure by ablating at least one nerve associated with the at least one anatomical structure. The unit is configured to be disposed in a catheter; and operating the catheter device 200 in the at least one anatomical structure, such that the at least one anatomical structure is substantially unharmed. The method 800 further comprises providing an imaging unit 602 configured to detect calcification, as indicated by block 820. The method 800 further comprises providing an expandable member 603 configured to be disposed at a distal end of the catheter 12, as indicated by block 830. Providing the imaging unit 602 comprises providing either the unit or the ablation unit with an imaging unit.

Still referring to FIG. 7, operating the catheter device 200, as indicated by block 840, comprises: using the imaging unit 602, detecting whether calcification is present in relation to the at least one anatomical structure; if calcification is present, inflating the expandable member 603, thereby enabling apposition of the expandable member 603 with at least one wall of the at least one anatomical structure; and using the decalcification unit to break/fragment the calcification; further inflating the expandable member 603 in relation to the at least one wall of the at least one anatomical structure; using the imaging unit 602, detecting whether the calcification is successfully broken in relation to the at least one anatomical structure; and if the calcification is successfully broken in relation to the at least one anatomical structure, using the ablation unit to denervate the at least one anatomical structure, such that the at least one anatomical structure is unharmed. In certain embodiments, calcification is determined to be sufficiently broken if the media-adventitia border is detectible, indicating that ultrasound waves are able to penetrate into the adventitia, past the calcification.

Still referring to FIG. 7, in the method 800, providing the expandable member 603, as indicated by block 840, further comprises disposing the expandable member 603 at a distal end of the catheter 12. Operating the catheter device 200, as indicated by block 840, further comprises, using the expandable member 603 to block at least one calcified region. The method 800 further comprises removing the at least one calcified region by aspirating the at least one anatomical structure. In the method 800, the at least one anatomical structure comprises at least one vasculature; and the at least one vasculature comprises at least one of: a renal vein and a renal artery. In some embodiments, the method of renal denervation may comprise advancing a catheter device 200 from an entry site on a patient to the target region of a vessel, e.g., renal artery with a calcified region. The catheter device 200 can comprise an ablation and/or decalcification transducer 500, ablation and/or decalcification electrode 510, and imaging transducer 520 along a catheter shaft 12a, and an expandable member 14. Once in the vessel, a calcified region can be identified using an imaging device. The imaging device may be configured for intravascular ultrasound (IVUS) imaging of the calcified region. The imaging device may be configured for identifying the calcified region of the renal artery. After the location and the calcified region is identified and the catheter device 200 is positioned adjacent to the calcified region, the expandable member 14 may be inflated by a fluid. The decalcification electrode 510 is activated to generate one or more bubbles adjacent to the calcified region to fragment the calcified region. Depending on the size of the calcified region, at least two decalcification electrodes 510 can be activated to generate one or more bubbles to fragment the calcified region. The progress of the fragmentation may be monitored by using the imaging device. Once the fragmentation is satisfactory, ablating one or more nerves within the calcified region or surrounding the calcified region or both is performed. After the ablation is complete, the expandable member 14 is deflated and the catheter device 200 is removed from the vessel. The imaging device may be separate from the catheter 12, or connected to the catheter 12. For example, the imaging device may be a separate system used in conjunction with the renal denervation catheter 12.

In some embodiments, the method of renal denervation may comprise advancing a catheter device 200 from an entry site on a patient to the target region of a vessel, e.g., a renal artery with a calcified region. The catheter device 200 may comprise an ablation and/or decalcification transducer 500, ablation and/or decalcification electrode 510, and imaging transducer 520 along a catheter shaft 12a, and an expandable member 14. Once in the vessel, a calcified region and a non-calcified region can be identified using an imaging device. The imaging device is configured for intravascular ultrasound (IVUS) imaging of the calcified region and non-calcified region. The location in the vessel, the calcified region, and non-calcified region are identified. The catheter device 200 is then positioned adjacent to the calcified region and the expandable member 14 may be inflated by a fluid. The decalcification electrode 510 is activated to generate one or more bubbles adjacent to the calcified region to fragment the calcified region. Depending on the size of the calcified region, at least two decalcification electrodes 510 can be activated to generate one or more bubbles to fragment the calcified region. The progress of the fragmentation may be monitored by using the imaging device. Once the fragmentation is satisfactory, ablating one or more nerves surrounding the renal artery is performed. After the ablation is complete, the expandable member 14 is deflated and the catheter device 200 is removed from the vessel.

In some embodiments, the method of renal denervation may comprise selecting a subject, e.g., patient with hypertension and one or more calcified regions associated with a renal artery. A catheter device 200 is advanced from an entry site on a subject to the target region of a vessel, e.g., renal artery with a calcified region. The catheter device 200 may comprise an ablation and/or decalcification transducer 500, ablation and/or decalcification electrode 510, and imaging transducer 520 along a catheter shaft 12a, and an expandable member 14. Once in the vessel, a calcified region and a non-calcified can be identified using an imaging device. The imaging device is configured for intravascular ultrasound (IVUS) imaging of the calcified region and non-calcified region. The decalcification electrode 510 is activated to generate one or more bubbles to the calcified region to fragment the calcified region. The progress of the fragmentation may be monitored by using the imaging device. Once the fragmentation is satisfactory, ablating one or more nerves within the identified non-calcified region of the renal artery or one or more nerves surrounding the identified non-calcified region of the renal artery or both. After the ablation is complete, the expandable member 14 is deflated and the catheter device 200 is removed from the vessel.

Referring to FIGS. 4A-7, in some embodiments of the present disclosure, the catheter device 200 is operable as a denervation device that identifies and breaks/fragments calcified plaques that are disposed on an arterial wall, verifies that the plaques have been removed, and applies ablative therapy. In some embodiments, the catheter device 200 is operable as a uRDN catheter. The catheter can include an imaging transducer to image at an optimal frequency of approximately 40 MHz, with a range frequency of approximately 20 MHz to approximately 60 MHz. The catheter device 200 can be configured to identify a calcium deposit.

Still referring to FIGS. 4A-7, in some embodiments of the present disclosure, the catheter device 200 comprises an imaging unit configured for intravascular ultrasound (IVUS) imaging having high acoustic reflection or scattering from calcification, whereby calcification is more readily detectable in an ultrasound image than otherwise by related art devices. In an embodiment, one or more steps or setbacks of the ablation transducer are used for imaging.

Still referring to FIGS. 4A-7, in an embodiment of the present disclosure, the catheter device 200 is operable as an uRDN catheter. The catheter device 200 further includes shockwave electrodes located distal and/or proximal to the transducer and spaced apart, such as approximately 3 mm to about 20 mm, e.g., approximately 5 mm, 6.7 mm, 7 mm, or 10 mm apart. The number of decalcification electrode 510 along the catheter shaft 12a may vary depending on the geometry of the target calcified region. For example, when it is intended for breaking up/fragmenting a calcified region along a long vessel segment, more decalcification electrodes 510 may be used along the catheter shaft 12a length, while a catheter device 200 intended for fragmenting a calcified region in a shorter vessel segment may have less decalcification electrodes 510 along the catheter shaft 12a length.

The decalcification electrode 510 may have a thickness from about 0.001 inch to about 0.01 inch, e.g., 0.002 inch, and may be attached along the catheter shaft 12a. There can be an insulating layer made of any material, e.g., Kapton, ceramic, polyimide, or Teflon, with a high breakdown voltage. The insulating layer may be about 0.001 inch to about 0.006 inch, e.g., 0.0015 inch, 0.0025 inch, and may have an opening that can be aligned over the decalcification electrode 510. In an embodiment, there can be a second decalcification electrode 510. The second decalcification electrode 510 may have a thickness from about 0.001 inch to about 0.015 inch, e.g., 0.0025 inch or 0.004 inch. The total thickness of the shock wave electrode assembly may be from about 0.002 inch to about 0.03 inch, e.g., 0.005 inch, 0.007 inch, or 0.008 inch. The required voltage comprises a range of approximately 100 Volts to approximately 10,000 Volts and depends on the size of the gap between the electrodes. In an embodiment, the generator may apply a voltage pulse such that the potential difference between the first decalcification electrode 510 and the second decalcification electrode 510 is high enough to form a plasma arc between them, generating a bubble that gives rise to a shock wave.

In an embodiment, the electrodes comprise metals such as stainless steel, tungsten, nickel, iron, steel, etc., such that they are configured to withstand high voltage levels and intense mechanical forces, e.g., in a range of approximately 1000 psi to approximately 2000 psi or in a range of approximately 20 atm to approximately 200 atm in a few microseconds, that are generated during use. The electrodes have a small surface area, such as to have a higher current density and, therefore, generate steam bubbles upon application of a high voltage. The generation, growth, and collapse of these bubbles produce cavitation and shock waves to break/fragment calcification. The direction of the resultant pressure pulse waves produced by the cavitation may be controlled based on the circumferential orientation of the electrode where cavitation is to occur. In an embodiment, the electrodes comprise two ring electrodes, each comprising an inner electrode, an insulating layer disposed over the inner electrode such that an opening in the insulating layer is aligned with the inner electrode, and an outer electrode sheath disposed over the insulating layer such that an opening in the outer electrode sheath is coaxially aligned with the opening in the insulating layer. In an embodiment, a shock wave generator, for example, decalcification electrodes 510, can be coupled to a high voltage source at the proximal end of the catheter device 200 via an electrical cable. When an expandable member 14, such as a balloon, is placed adjacent to a calcified region of a blood vessel, such as a renal artery, a low voltage is applied across the decalcification electrodes 510 for about 2 milliseconds (ms) to ensure that an arc does not form across the decalcification electrodes 510. During this 2 ms period, a bubble is formed on one of the decalcification electrodes 510. The bubble size may be controlled by the amount of current and the length of time the low current is applied. After the 2 ms period, a narrow pulse (500 nanoseconds (ns)) is applied of the full 3,000 volt high voltage across the decalcification electrode 510.

Still referring to FIGS. 4A-7, in an embodiment of the present disclosure, the electrodes comprise two simple electrodes (+ and −) distal and proximal the ablation transducer (or the ablation transducer/imaging transducer) that are added to the ablation system 100. The voltage may be adjusted correctly based on the distance, so that an electrical arc can be introduced to generate high temperature (just local area) and vapor bubbles in the fluid. In certain embodiments, multiple electrodes (an array) can be used. The electrodes 510 may be placed at a controlled distance apart on the catheter shaft 12a to allow a reproducible arc for a given voltage and current. The electrical arcs between the electrodes 510 in the fluid can be used to generate shock waves in the fluid. The generator can be a variable high voltage pulse generator configured to deliver a stream of pulses to the decalcification electrodes 510 to create a stream of shock waves within the expandable member 14 and within the blood vessel being treated. The magnitude of the shock wave can be controlled by controlling the magnitude of the pulsed voltage, the current, the duration, and repetition rate.

Still referring to FIGS. 3-7, in an embodiment of the present disclosure, the expandable member, e.g., a balloon, comprises an electrically insulating material, such as polyamide, polyethylene terephthalate, or thermoplastic elastomer. In specific embodiments the expandable member comprises nylon, a polyimide film, a thermoplastic elastomer (such as those marked under the trademark PEBAX™), a medical-grade thermoplastic polyurethane elastomers (such as those marketed under the trademark PELLETHANE™), pellethane, isothane, or other suitable polymers or any combination thereof, but is not limited thereto. The length of the balloon may vary depending on the number of decalcification electrodes 510 present.

Still referring to FIGS. 4A-7, in an embodiment of the present disclosure, power is applied to form gas bubble at the surface of the electrodes, thereby causing a plasma arc of electric current to traverse the bubble, thereby generating a rapidly expanding and collapsing bubble, which, in turn, effects a mechanical shock wave in the balloon. The shock waves mechanically conduct through the fluid and through the expandable member to apply mechanical force or pressure to break apart/fragment any calcified plaques on, or in, the vasculature walls. In some embodiments, the shock wave can radiate outward from the decalcification electrode 510 in all directions and travel through the expandable member 14, to the blood vessel where the decalcification electrode 510 can fragment the calcified region. In some embodiments, the first and second decalcification electrode 510 may be located radially across from each other such that the shock waves they each generate propagate in opposite directions. The shock waves generated by each of the decalcification electrode 510 may propagate outward, with an angular spread of about 180 degrees. In some embodiments, the decalcification electrode 510 may generate shock waves that propagate from the left and right longitudinal side of the catheter shaft 12a, while other decalcification electrodes 510 may generate shock waves that propagate from the top and bottom longitudinal side of the catheter shaft 12a.

In some embodiments, the decalcification electrode 510 may generate a pair of shockwaves that propagate outward from positions at 0 degrees and 180 degrees around the circumference of the catheter shaft 12a. In some embodiments, the decalcification electrode 510 may generate a pair of shockwaves that propagate outward from positions at 60 degrees and 240 degrees around the circumference of the catheter shaft 12a. In some embodiments, the decalcification electrode 510 may each generate a pair of shockwaves that propagate outward at the same locations around the circumference of the catheter shaft 12a, but from different locations along the length of the catheter shaft 12a.

Still referring to FIGS. 4A-7, in an embodiment of the present disclosure, intermittent sound waves that are emitted by using the electrodes comprise sequential pulses with multiple cycles, e.g., approximately 8 cycles to approximately 12 cycles at a fixed pulse repetition rate, such as 1 pulse per second (the pulse duration is dependent on the surface area of the electrodes) to create acoustic pressure waves that produce a circumferential field effect to fracture calcification. Prior to the application of the shockwave, the expandable member is inflated at a pressure to achieve apposition against the arterial wall, e.g., 1 atm or 4 atm. The expandable member can then be inflated to a higher pressure as calcification(s) is/are fractured.

Still referring to FIGS. 5A-7, in an embodiment of the present disclosure, a 50/50 ratio saline solution to contrast medium is used for inflation of the expandable member during admission of the shockwave. Other conductive fluids may be used instead of a saline solution. The ultrasound transducers can be either insulated or non-insulated from the fluid. The conductivity of the fluid can be adjusted to facilitate formation of gas bubbles (cavitation). In certain embodiments, the saline solution comprises a salt content between about 0.9 weight percent (wt. %) and about 5 wt. %. The higher the salt content of the conductive fluid, the higher the conductance will be for the fluid, thereby requiring less energy to increase the temperature of the fluid and induce the formation of bubbles. In certain embodiments, a contrast medium may be avoided by using a transducer unit for imaging.

Still referring to FIGS. 4A-7, in some embodiments, a decalcification transducer unit is used instead of, or in combination with, shockwave electrodes. The decalcification transducer may comprise a third transducer unit optimized for inducing cavitation, e.g., a piezoelectric focused transducer with an operable frequency in a range of approximately 400 kHz to approximately 3 MHz. The third transducer unit is configured to break/fragment calcium deposits from the arterial wall without substantially damaging the arterial wall. In an embodiment, there may be a series of ultrasound transducers that may be operated simultaneously to decalcify. In an embodiment, there may be a series of ultrasound transducers that may be operated independently to decalcify the calcified region. In some embodiments, the transducer 500 may be intermittently operated at a relatively low and a relatively high frequency to cavitate and ablate, respectively. This may be achieved by operating the transducer 500 in its relatively low harmonic and its relatively high harmonic. Alternatively, this may be achieved by configuring different transducers 500 to have a different resonant frequency at the first harmonic. For each transducer 500 to deliver different amounts of decalcification and/or ablative energy, the amount of power delivered to each transducer 500 may be different. Therefore, in some embodiments, the transducer 500 may be wired separately and in a different manner. This may allow energizing each of the transducers 500 separately giving more control to the user. In an embodiment, one or more transducers 500 may be configured to rotate using a rotating unit (not shown).

Still referring to FIGS. 4A-7, in some embodiments, a flexible post (embodiments shown in FIGS. 3A-3C) may separate the unfocused ablation and focused decalcification transducer units in order to facilitate delivery of the catheter device 200. In certain embodiments, the catheter device 200 may comprise an imaging transducer, ablation transducer, decalcification transducer, and treatment confirmation electrodes coupled with a balloon in a manner that does not interfere with sonication of the transducers.

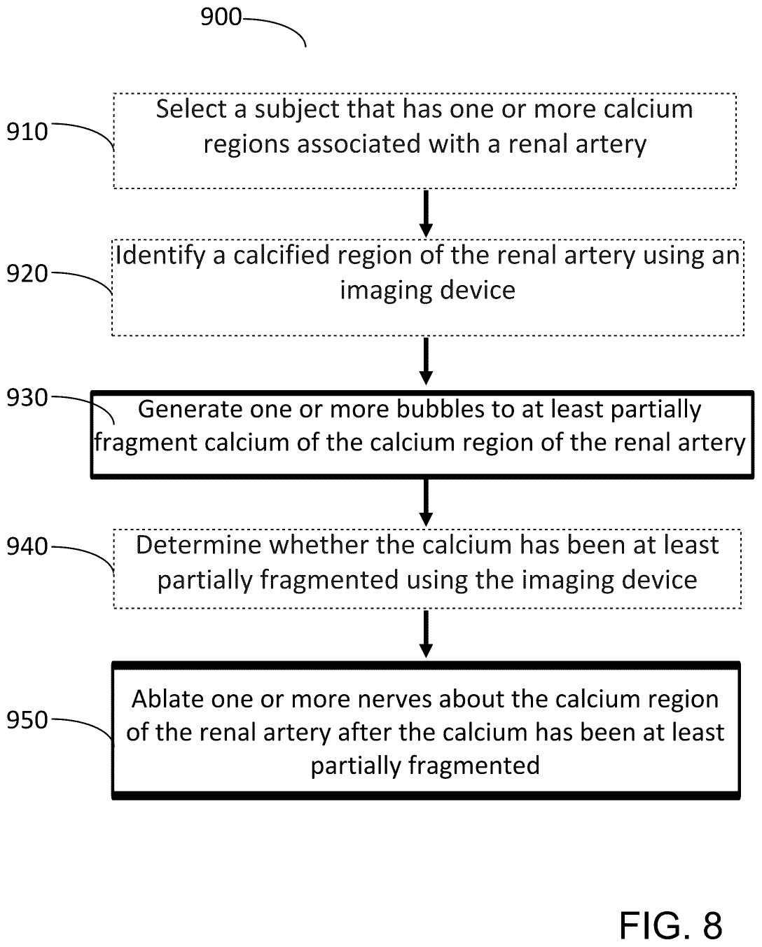

FIG. 8 is an example of a block diagram illustrating a process 900 of pretreating a calcified region prior to ablating one or more nerves about the calcified region described above with reference to FIGS. 1-8, in accordance with an embodiment of the present disclosure. The method 900 can optionally include selecting a subject that has one or more calcified regions associated with a renal artery 910. The method 900 may also optionally include identifying a calcified region of the renal artery using an imaging device 920.

In some embodiments, the method 900 may include, e.g., at operation 930, generating one or more bubbles to at least partially fragment calcification of the calcified region of the renal artery. In some embodiments, vapor bubbles may be generated. Various types of bubbles, e.g., microbubbles, gas bubbles, or steam bubbles, etc., may be used so long as there is sufficient energy to fragment calcification. Generating one or more bubbles may be done using decalcification electrodes 510 or a decalcification transducer 500 as described.

In some embodiments, the method 900 may include, e.g., at operation 940, determining whether the calcification has been at least partially fragmented using the imaging device. Alternatively, the process 900 may be done without determining whether the calcification has been partially fragmented.

Once the calcified region has been at least partially fragmented, the one or more nerves about the calcified region of the renal artery may be ablated at operation 950. The targeted nerves for ablation may be within and/or surround the calcified region or nearby the calcified region. When the nerves are located nearby the calcified region, the user may move the catheter device 200 along the renal artery to a different location after decalcification to ablate the nerves.

Calcification including its shape, location, content, and surrounding structures is usually well seen under ultrasound imaging, such as an independent or integrated IVUS transducer on the ablation catheter. With other technologies, such as OCT or ultrasound imaging using super high frequencies (greater than 40 MHz), when penetration is challenging, visualizing the media-adventitia border using an imaging device may be a method used to confirm calcification/decalcification. In other words, if the media-adventitia border cannot be seen by the imaging device, then a calcified region is most likely present. However, if the media-adventitia border is seen by the imaging device, then a calcified region is most likely not present or there is a very thin layer of calcification present.

In the description above, pretreatment is effected by one or more decalcification units used to decalcify an anatomical structure. The decalcification unit(s) can include, by way of example, the decalcification electrodes 510 or the decalcification transducer 500. It will be appreciated, however, that such examples are not limiting of the range of decalcification units that may be incorporated in the methods described above. For example, in an embodiment, pretreatment may be done using a laser. More particularly, the decalcification unit(s) can include a laser to decalcify the calcified region prior to treatment with a transducer 16. The laser may include a holmium:yttrium-aluminum garnet (Ho:YAG), thulium fiber laser (TFL), or thulium:yttrium-aluminum garnet (Tm:YAG) laser.

In an embodiment, the laser can include a holmium:yttrium-aluminum garnet (Ho:YAG) laser. The Ho:YAG laser may be used to decalcify a calcified region by vaporization. The holmium infrared laser wavelength of 2100 nm is highly absorbed by water (water absorption coefficient of 3198 L/m). The water absorbed energy results in the formation of a vapor microbubble at the tip of the laser that expands outwardly toward the target; once the microbubble reaches the target, the laser beam can pass through the vapor to the target with little attenuation because the density of the water molecules in the steam is much less than in the liquid state. The bubble can be initiated with a very small amount of energy, and the threshold for bubble formation (100-200 ms) and expansion is independent of the duration or excess energy in the pulse. The Ho:YAG laser can produce smaller plaque fragments.

The laser pulses of the Ho:YAG system can have an average power of about 120-140 W, a pulse frequency of about 5-80 Hz (and up to 120 Hz), a pulse energy of about 0.2-6.0 J, a pulse width of about 50-1300 μs (which may be adjustable (short, medium, and long)), and a silica fiber of about ≥200 μm.

In an embodiment, the laser can include a thulium fiber laser (TFL). The TFL may be used to decalcify a calcified region. With TFL, multiple electronically modulated laser diodes can be used to excite thulium ions for laser pumping, instead of the flash lamps used in Ho:YAG lasers. The emitted laser beam can have a wavelength of 1940 nm, which can perform in a continuous or pulsed mode. The laser beam can be more uniform and focused, as compared to a Ho:YAG laser beam, and can be transmitted to smaller core diameter fibers (50-150 mm). TFL provides low (e.g., as low as 0.025 J) to high (e.g., as high as 6 J) pulse energies, high pulse frequencies (up to 2400 Hz for the latest TFL device), short to long pulse durations (200 ms-50 ms), peak power of 500 W, and average power of 2 to 60 W. The TFL system can have an average power of about 2-60 W, a pulse frequency of about 1-2400 Hz, a pulse energy of about 0.025-6.0 J, a pulse width of about 200 μs-50 ms, a peak power of about 500 W, a silica fiber of about ≥50 μm.

In an embodiment, the laser can include a Tm: YAG laser. The Tm: YAG laser can be a solid-state laser, may be used to decalcify a calcified region by a photo-thermal mechanism. Tm: YAG provides 120 W of power with frequencies of 1 to 200 Hz and possible pulse energies as low as 0.1 J up to 3 J. The Tm: YAG system can have an average power of about 120 W, a pulse frequency of about 1-200 Hz, a pulse energy of about 0.1-3 J, a peak power of about 200 W, and a silica fiber of about 400 μm.

In certain embodiments, the laser fiber disclosed herein may, in addition to decalcifying the calcified region, be used to ablate nerve fibers about, within, and/or surrounding the body lumen. For example, the laser fiber can direct energy into an arterial wall to act in addition to or in replacement of the ultrasound transducer. More particularly, the energy can ablate the never fibers about, within, or surrounding the calcification. Additional description of the catheter incorporating a laser for decalcification and ablation is described below.

In an embodiment, a laser fiber 302 is located on a balloon surface, e.g., a shoulder, a bottom, a top, a front, a back or a center of the balloon 14. The laser fiber 302 can fire in any direction, e.g., front, back, top, bottom, center, or at an angle. More particularly, a front-firing laser fiber can be oriented such that laser radiation emits in a distal direction, e.g., parallel to a body lumen wall. By contrast, a side-firing laser fiber may be oriented such that laser radiation emits radially outward, e.g., perpendicular to the body lumen wall. Decreasing the pre-treatment time or circulating cooling fluid adjacent to where the laser fiber 302 is located are some methods of lowering heat generated by the laser. The laser fiber 302 is energized by a source, e.g., generator.

In several of the embodiments described below, there is at least one laser fiber 302 on the balloon surface. For example, the laser fiber 302 can be mounted on the surface of the balloon. The laser fiber 302 can be located close to the calcified region when the balloon 14 is at least partially inflated. For example, a distance between the laser fiber 302 and the body lumen wall, e.g., an arterial wall, can be in a range of about 0.1 to 1 mm. The laser fiber 302 can be disposed adjacent to, but not necessarily contacting, the calcification of the calcified region of the body lumen wall before cavitation.

As described above, the catheter 200 can include an imaging device. In an embodiment, the imaging device includes the laser fiber 302. More particularly, the laser fiber 302 may be used to image/visualize the calcified region and/or arterial lumen. The laser fiber 302 may also be used to image/visualize balloon apposition against the body lumen wall. Changing the frequency allows the laser fiber 302 to be used for decalcification and/or imaging. The laser fiber 302 may have a lens for imaging in addition to generating one or more bubble to cause cavitation to fragment the calcified region. In an embodiment, the laser fiber 302 may be a light illuminating fiber for viewing the catheter device 200 or calcified region. For example, the laser fiber can include a fiber optic cable to deliver light that is reflected into the lens and returned to an imaging system for imaging. All parts insides the balloon including the balloon surface can be entirely or partially covered with fluorescent materials to illuminate the vessel walls. When the optical fiber/laser fiber 302 is used to examine vessel walls, the light source may be another laser fiber or a fluorescent light source. The illuminating light may be monochromic and the image may be viewed using a corresponding laser fiber/camera that may be separate from the catheter device 200 or mounted on the catheter balloon. The laser fiber 302 runs from the energy source e.g., generator and through the catheter shaft 12a before it mounts on the balloon 14. The laser fiber 302 may be mounted to the surface of the balloon 14 with an adhesive, e.g., bonding with flexible UV-adhesives with increased elongation, or the laser fiber 302 may be embedded into the balloon 14 by lamination during the balloon blow-molding process.

In an embodiment, the laser fiber 302 can run parallel with the balloon surface. The laser fiber 302 may be flexible to prevent breaking. Decalcification of the calcified region may be local at a target region or circumferential. In embodiments where there is only one laser fiber 302, the catheter device 200 may be rotated or twisted to inspect the body lumen for calcified regions. Rotating or twisting the catheter device 200 may be done manually or by using a drive cable.

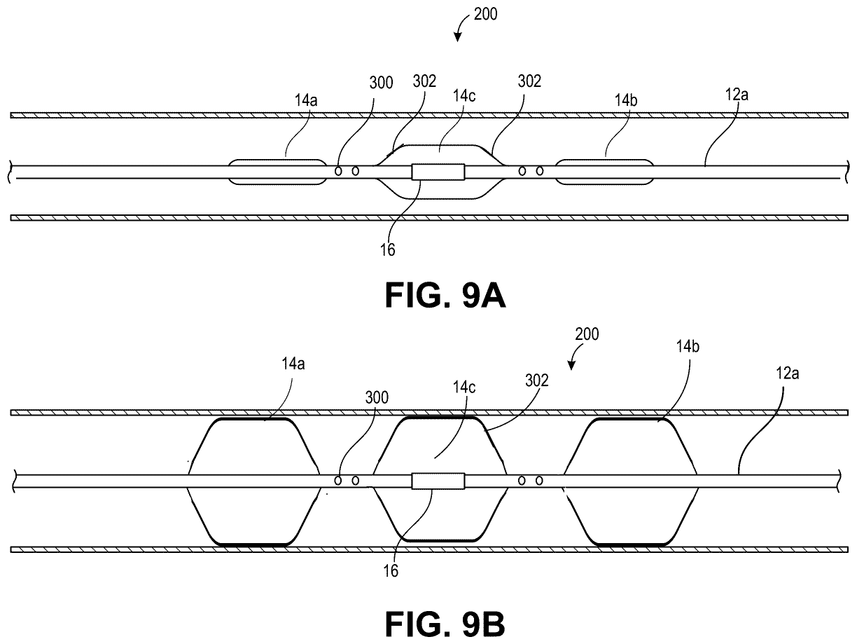

FIG. 9A is a diagram illustrating a side view of one embodiment of a catheter device 200 having a deflated proximal balloon 14a and distal balloon 14b for blocking debris after pretreating. Balloons 14a, 14b, and 14c are separate balloons and are controlled independently from one another. For example, respective interiors of the balloons can be in fluid communication with respective fluid lumens running through a catheter shaft 12a. Inflation fluid may therefore be conveyed through the fluid lumens to separately inflate or deflate the balloons.

The laser fiber 302 may be located on a surface of the expandable member 14C, e.g., a balloon. For example, the laser fiber 302 can be mounted such that an outlet, e.g., a port through which laser emits from the laser fiber into the surrounding environment, is on a proximal shoulder or a radially outward surface of the balloon. Such location can orient the outlet to fire a laser beam longitudinally forward in a direction of the body lumen, or radially outward toward a body lumen wall of the body lumen.

Transducer 16 can be located inside the balloon 14c. Imaging may be performed, in addition to or instead of the laser fiber 302, by the transducer 16. More particularly, the transducer 16 can image or detect calcification as described above with respect to, e.g., FIG. 5. It will be appreciated that similarly, the laser fiber 302 may be incorporated into embodiments described above to perform such detection.

In an embodiment, the balloons 14a, 14b, and 14c are mounted on the catheter shaft 12a. The balloons can be located on the catheter shaft in sequence. More particularly, a middle balloon 14c can be longitudinally between the proximal balloon 14a and the distal balloon 14b. Accordingly, when the middle balloon 14c is longitudinally aligned with a calcification of the calcified region of the body lumen, the calcification can be longitudinally between the proximal balloon 14a and the distal balloon 14b.

In an embodiment, the catheter 200 includes debris removal feature. Debris generated during a decalcification process, e.g., fragments or calculi of the calcification, can lead to emboli that may place a patient at risk. Accordingly, catheter 200 can include the debris removal feature to capture and/or remove the debris from the patient anatomy. In an embodiment, the debris removal feature includes one or more holes 300 in the catheter shaft 12a. Suction may be applied through the hole(s) 300 to retract debris into the catheter shaft 12a. The suctioned debris may therefore be removed from the patient anatomy without forming emboli that could pass distally beyond the treatment area and create an emboli risk.

FIG. 9B is a diagram illustrating a side view of the catheter device 200 having an inflated proximal balloon 14a and distal balloon 14b for blocking debris after pretreating. In an embodiment, an imaging device may be separate from the catheter device 200. If an imaging device, e.g., IVUS, OCT, are separate from the catheter device 200, then the imaging device can first be inserted into the body lumen (BL) to image the wall of the body lumen, e.g., renal artery. Alternatively, the imaging device can be integrated with the laser fiber and or incorporate the transducer, as described above. The imaging device, whether integrated or separate from the catheter 200, allows the user to visualize a calcified region. When a calcified region is detected, the imaging device is removed from the body lumen (if the imaging device is separate) and the catheter device 200 with deflated balloons 14a, 14b, and 14c, is delivered adjacent to the calcification of the calcified region of the body lumen wall. After positioning the catheter device 200 adjacent to the calcified region, the suction device is turned on via the generator. The suction retracts or sucks fluid, and debris within the fluid, from the body lumen into the hole(s) 300. Suction may be turned on/off at any time during pre-treatment.

As shown in FIG. 9B, the proximal balloon 14a, middle balloon 14c, and distal balloon 14b can be inflated using a circulating fluid. Once the balloons 14a, 14b, 14c reach apposition, the laser fiber 302 can be energized. Laser emitted from the laser fiber can generate one or more bubbles to break apart at least a part of the calcified region. For example, energy absorbed by water within the blood at the treatment site can generate bubbles within a zone 0.5 to 1.0 mm from the body lumen wall. Creating bubbles within the zone adjacent to the body lumen wall can localize the cavitation shock waves to loosen and/or fragment the calcification. Some of the broken up debris may remain within the body lumen wall, e.g., at the media-adventitia interface or within the media layer. Alternatively, some of the broken up debris may release into the body lumen, e.g., radially inward of the intima.

The proximal balloon 14a and the distal balloon 14b can contain released debris in an area where the holes 300 are located to suction away the debris. The balloons 14a, 14b, 14c may be deflated and the catheter device 200 may be withdrawn from the body lumen.

Following fragmentation and debris removal, the imaging device can again be used to image the calcified region. For example, a separate imaging device may be re-inserted into the body lumen. Alternatively, the laser fiber can be used in an imaging mode to capture images and/or reflected radiation that may be analyzed to image/confirm that the calcified region has been appropriately decalcified. If it is confirmed that the calcified region was not appropriately fragmented, then the process previously described may be repeated. However, if it is confirmed by the imaging device that the calcified region was appropriately decalcified, the catheter device 200 can be used to ablate nerves about, within, or surrounding the calcified region.

Ablation can begin by re-inserting, delivering, or otherwise disposing the catheter 200 in the body lumen such that the transducer 16 is placed near the region of decalcification. The balloon 14c can be inflated using a circulating fluid to obtain apposition with the lumen wall. When apposition is obtained, the transducer 16 can be energized to denervate the nerve surrounding the body lumen. In another embodiment, balloons 14a and 14b may be inflated first to block the proximal end and the distal end of the body lumen. Thereafter, balloons 14c may be inflated to bring the laser fiber 302 adjacent to the calcified region for decalcification. Balloons 14a, 14b, and 14c may be deflated concurrently or separately.

In another embodiment, the imaging device may be integrated with the catheter device 200. In such an embodiment, the catheter device 200 is inserted into the body lumen and the imaging device is used to locate a calcified region. The laser fiber 302 may also function as an imaging device. When a calcified region is located, balloons 14a, 14b, 14c are inflated adjacent to the calcified region to obtain apposition with the wall of the body lumen. During this process, the suction may be turned on or off. Once the balloons 14a, 14b, 14c reach apposition, the laser fiber 302 is energized and calcified region will be at least partially fragmented. Balloons 14a, 14b may be inflated first to block the proximal and distal ends of the body lumen and balloon 14c may be inflated last. The proximal balloon 14a and the distal balloon 14b can block the debris from floating away. During decalcification, the suction from the hole 300 can be on and the suction removes the calcium debris that is contained by balloons 14a, 14b. In an embodiment, the catheter shaft 12a has multiple lumens for wires, suction, and fluid flow. In an embodiment, the inflation/deflation of the balloons 14a, 14b, 14c may be controlled independently.

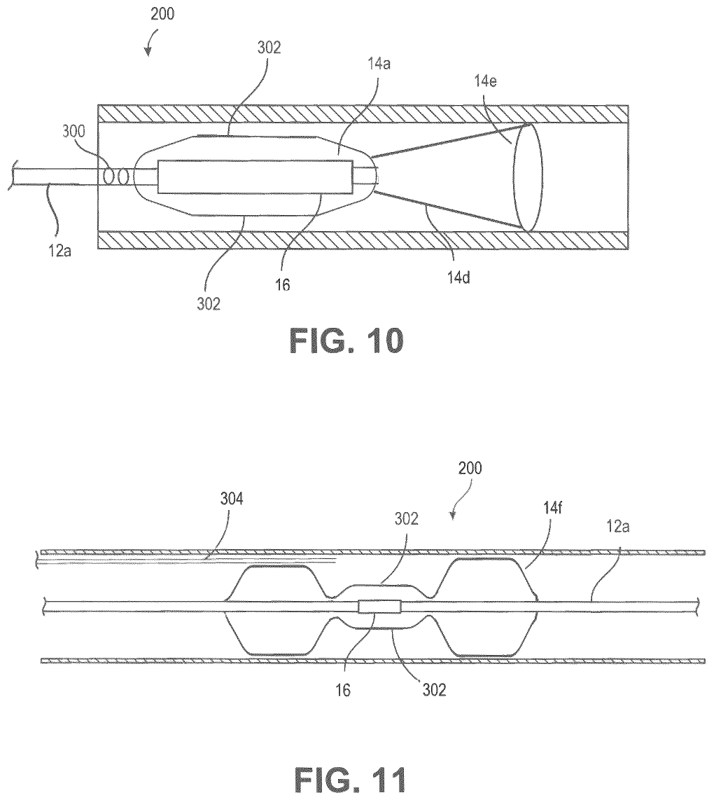

Referring to FIG. 10, a diagram illustrating a side view of a catheter device having a cone-shaped balloon is shown. A cone-shaped balloon 14e can be mounted on a catheter shaft 12a of the catheter 200. The cone-shaped balloon can be located at a distal portion of the catheter device 200 for blocking debris after pretreating. The cone-shaped balloon 14e can comprise an outer, tapered surface 14d which slowly move toward the lumen wall after the distal portion reaches apposition during inflation. More particularly, the surface 14d can be conical such that a distal end of the surface has a larger cross-sectional diameter than a proximal end of the surface.

The catheter 200 may be deployed and used in a sequence similar to that described above with respect to FIGS. 9A-9B. After the calcified region is fragmented with the laser fiber 302, which may be mounted on a balloon 14a such that an outlet of the laser fiber generates one or more bubbles near a body lumen wall, the debris from the calcified region can be contained. Capture of the debris can be achieved by suctioning fluid through the hole(s) 300 in the catheter shaft 12a. The suction can cause the outer surface 14d of the cone-shaped balloon 14e to fold back and splay outward against the lumen wall. In other words, the distal portion of the cone-shaped balloon 14e blocks the debris from moving distally because it reaches apposition with the vessel wall to block distal flow of debris when decalcification occurs. Towards the end or at the end of the decalcification operation, the cone-shaped balloon 14e slowly inflates moving the sides 14d of the balloon 14 to approach apposition against the lumen wall. This forces the debris from the decalcification to move in a proximal direction and directs the debris towards the hole 300 for suction. Once the entire cone-shaped balloon 14e reaches apposition, the debris is located proximally near the hole 300 for suction. The risk of emboli flowing downstream in the body lumen is thereby mitigated.

As shown in FIG. 10, the laser fiber 302 can be located on an upper and lower surface of the proximal balloon 14a. More particularly, several laser fibers may be used having respective outlets at diametrically opposed locations on a radially outward, working length of the balloon 14a. The laser fiber 302 may also be located on the shoulder of the proximal balloon 14a. The laser fiber 302 may be located in different locations on the surface of the proximal balloon 14a. The inflation of the proximal balloon 14a and the cone-shaped balloon 14e may be controlled separately using circulating fluid that moves through the fluid lumens located in the catheter shaft 12a.

One method for pre-treating/treating comprises imaging the body lumen before pretreatment and treatment; however, imaging is optional. An imaging device may be separate from the catheter device 200. If an imaging device, e.g., IVUS, OCT, are separate from the catheter device 200, then the imaging device is first inserted into the body lumen to image the wall of the body lumen, e.g., renal artery. When a calcified region is detected, the imaging device is removed from the body lumen and the catheter device 200 with deflated balloons 14a, 14e is inserted into the body lumen. After positioning the catheter device 200 adjacent to the calcified region, the suction device is turned on via the generator. The suction is felt through the hole 300. However, suction may be turned on/off at any time during treatment. As shown in FIG. 10, the proximal balloon 14a and cone-shaped balloon 14e are inflated using a circulating fluid flowing through the fluid lumens located in the catheter shaft 12a. Once the distal portion of the cone-shaped balloon 14e reaches apposition, proximal balloon 14a inflates to move the laser fiber 302 closer to the calcified region. The laser fiber 302 is energized to at least partially fragment the calcified region and the proximal balloon 14a is deflated. The proximal balloon 14a and the cone-shaped balloon 14e contains the debris in an area where the holes 300 are located to suction away the debris. The balloons 14a and 14e may be deflated and the catheter device 200 may be withdrawn from the body lumen to allow the imaging device to be re-inserted into the body lumen to image/confirm that the calcified region has been appropriately decalcified. If it is confirmed that the calcified region was not appropriately decalcified, then the process previously described may be repeated. However, if it is confirmed by the imaging device that the calcified region was appropriately decalcified, the catheter device 200 is re-inserted into the body lumen and placed at the region of decalcification. The proximal balloon 14a can be inflated using a circulating fluid to obtain apposition with the lumen wall. When apposition is obtained, the transducer can be energized to denervate the nerve surrounding the body lumen.

In another embodiment, the imaging device may be integrated with the catheter device 200. In such an embodiment, a method for pre-treating/treating comprises inserting catheter device 200 into the body lumen. The integrated imaging device, e.g., laser fiber 302 is used to locate a calcified region. When a calcified region is located, the distal portion of the cone-shaped balloon 14e is inflated adjacent to the calcified region to obtain apposition with the wall of the body lumen. During this process, the suction may be turned on or off. Once the distal portion of the cone-shaped balloon 14e reaches apposition, the proximal balloon 14a is inflated to bring the laser fiber 302 closer to the calcified region. Then the laser fiber is energized and calcified region will be at least partially decalcified. The proximal balloon 14a is slightly deflated and the cone-shaped balloon 14e slowly inflates proximally pushing the debris towards the hole 300 to be suctioned at the proximal end. After the debris is removed, the proximal balloon 14a inflates to reach apposition against the lumen wall. Thereafter, the transducer 16 is energized to denervate surrounding nerves. In an embodiment, the catheter shaft 12a has multiple lumens for wires, suction, and fluid flow.

Referring to FIG. 11, a diagram illustrating a side view of a catheter device is shown. The catheter 200 includes a barbell-shaped balloon 14f mounted on the catheter shaft 12a. The barbell-shaped balloon 14f can block debris after pretreating. The barbell-shaped balloon 14f inflates at a proximal region and a distal region. The proximal and distal regions can inflated before a middle region of the barbell-shaped balloon 14f inflates. The middle region can surround the transducer 16.

In an embodiment, the barbell-shaped balloon 14f is an only balloon 14 of catheter 200, and the imaging device may be separate or integrated with the catheter device 200. The laser fiber 302 can be disposed on the surface of the barbell-shaped balloon 14f. One method for pre-treating/treating comprises inserting the imaging device into the body lumen to image the wall of the body lumen, e.g., renal artery. When a calcified region is detected, the imaging device is removed from the body lumen and the catheter device 200 with deflated barbell-shaped balloon 14f is inserted into the body lumen. After positioning the deflated barbell-shaped balloon 14f adjacent to the calcified region, a separate suction device 304 can be inserted into the body lumen near the region of the laser fiber 302. The suction may be turned on/off at any time during treatment. As shown in FIG. 11, the barbell-shaped balloon 14f can be inflated using circulating fluid. The ends, e.g., the proximal region and the distal region, of the barbell-shaped balloon 14f can reach apposition first and then the middle where the laser fiber 302 is located. When the laser fiber 302 is close to the wall of the body lumen, the laser fiber 302 can be energized. The laser fiber can emit laser energy to generate one or more bubbles that at least partially fragment or break apart the calcified region. During this process, the debris can be removed by the suction device 304. For example, the suction device can be an external tubular element that is placed between the barbell-shaped balloon 14f and the body lumen wall. An inlet of the tubular element can be disposed within a volume defined between the body lumen wall and the balloon, e.g., longitudinally between the proximal region and the distal region of the barbell-shaped balloon 14f. The proximal and distal regions of the barbell-shaped balloon 14f can contain the debris in the area where the suction device 304 opening is located. The debris can therefore be removed from the area to reduce a risk of embolism. Thereafter, the transducer 16 can be energized, as described above, to denervate surrounding nerves.

The external tubular element may be used in the embodiments described above. Similarly, the hole(s) 300 described in the embodiments above may be incorporated into the catheter 200 of FIG. 11 to remove debris from the treatment area. Accordingly, it will be appreciated that features of catheter systems described throughout this disclosure can be interchangeable in certain embodiments.

Referring to FIG. 12, a side view of one embodiment of a catheter device having at least one laser fiber is shown. The at least one fiber laser 302 can be located on the surface of the balloon 14. The catheter 200 can include a scoop 306 for collecting debris from the decalcification. The scoop 306 may be deployed at a distal region of the catheter device. The laser fiber 302 can be located on the surface of the balloon 14, as described above. For example, the laser fiber 302 may be located on a distal shoulder of the balloon 14, and can direct energy into the body lumen such that one or more cavitation bubbles are generated near the body lumen wall. The cavitation creates shock waves to pulverize the calcification, and fragments the calcified region may be released into the body lumen. In an embodiment, the scoop 306 collects the debris in the body lumen (BL) before the transducer 16 denervates the nerves in the surrounding area. The scoop 306 can collect the debris when the catheter device 200 is pulled from the distal to proximal direction. An imaging device may be separate or integrated with the catheter device 200 shown in FIG. 12.

One method of pre-treating/treating comprises first inserting the separate imaging device into the body lumen (BL) to image the wall of the body lumen, e.g., renal artery. When a calcified region is detected, the imaging device is removed from the body lumen and the catheter device 200 is inserted into the body lumen. After positioning the catheter device 200 adjacent to the calcified region the scoop 306 is deployed and the balloon 14 is inflated so that the laser fiber 302 is adjacent to the calcified region. Once the laser fiber 302 is adjacent to the calcified region, the laser fiber can be energized to cavitate the calcified region. The scoop 306 can catch the debris and the user may remove the catheter device 200 by pulling the catheter device 200 proximally. The user may re-insert the imaging device to visualize whether the calcified region or the media-adventitia border is visible. If it is determined that the media-adventitia border is visible or that the calcified region has been decalcified, the image device is removed and the catheter device 200 is reinserted to begin treatment. The user can reinsert the catheter device 200 adjacent to the decalcified region, inflate the balloon, and energize the ultrasound transducer to ablate the nerves surrounding the area.

In an embodiment, where the imaging device is integrated with the catheter device 200, the laser fiber 302 can image and cavitate depending on the frequency at which it runs. The user can insert the catheter device 200 into the body lumen and energize the laser fiber 302 to visualize the lumen and/or calcified region. Once the calcified region is found, the scoop 306 can be deployed and the balloon 14 can be inflated so that the laser fiber 302 is adjacent to the calcified region. The laser fiber 302 is then energized to cavitate the calcified region and the scoop catches the debris. Once it is determined that the calcified region has been appropriately decalcified via the imaging laser fiber 302, the ultrasound transducer 16 is energized to ablate the surrounding nerves. Once ablation is completed, the user can withdraw the catheter device 200.

Other embodiments to prevent fragments from causing emboli may be employed. For example, an embolic protection device separate from the catheter 200 can be used to provide distal vessel protection. The embolic protection device can include a separate balloon mounted on a wire, or a filter basket. The embolic protection device can be advanced distal to the calcified region. The catheter 200 may be delivered to the calcified region, e.g., over the wire of the embolic protection device, to fragment the calcification and ablate the nervous tissue. Dislodged calculi can be captured and retrieved by the embolic protection device. Other embodiments for decalcification of a calcified region may be employed, e.g., rotational atherectomy, orbital atherectomy, cutting, scoring, or sculpting balloons.

In the embodiments described above, the laser fiber 303 can be mounted on one or more of the balloons of the catheter 200. The laser fiber can direct energy into the region around the balloon and, thus, the balloon may absorb some of the energy and become heated. In an embodiment, an insulative or reflective element is used to protect the balloon material from overheating. For example, an insulation sleeve can be located over the surface of the balloon, between the balloon and the outlet of the laser fiber, to insulate the surface form the laser energy. Similarly, a foil can be located between the laser fiber outlet and the balloon surface to reflect and direct laser radiation away from the balloon. The balloon material may thereby be insulated and protected from laser-generated heat that could compromise, e.g., melt, the balloon material.

In some embodiments, the method of ablating a renal nerve of a subject using the catheter device 200 may include emitting an energy to the renal nerve from a location within the renal artery. The energy emitted may be acoustic energy, e.g., ultrasound. An imaging device, e.g., x-rays, ultrasonography and non-contrast CT, PET scans, extravascular ultrasound, intravascular ultrasound, MRI, OCT, IVUS, or angiography, may be used to identify whether there is a calcification about and/or surrounding the location within the renal artery. The ablation transducer 500 may ablate the renal nerve when the location is non-calcified or when a calcium score of the location is low.

In some embodiments, the method of ablating a renal nerve of a subject with an ablating device may include emitting an energy, e.g., acoustic energy, to the renal nerve from a location within a renal artery. Identifying whether there is a calcification about and/or surrounding the location within the renal artery may be done by using an imaging device, such as an imaging transducer 520. Decalcification occurs by generating one or more bubbles to fragment calcification at least partially at the location where the location is calcified. An ablation transducer is used to ablate the renal nerve after the calcification has been at least partially fragmented. In some embodiments, decalcification may not occur due to the lack of calcification. As such, decalcification may be skipped, and ablating may be done instead.

FIG. 15 will now be used to describe an example implementation of the controller 1500. Referring to FIG. 15, the controller 1500 is shown as including one or more processors 1512, a memory 1514, a user interface 1516, and an ablation unit excitation source 1518, a decalcification unit excitation source 1520, but can include additional and/or alternative components. While not specifically shown, a processor 1512 can be located on a control board, or more generally, a printed circuit board (PCB) along with additional circuitry of the controller 1500. The processor 1512 can communicate with the memory 1514, which can be a non-transitory computer-readable medium storing instructions. The processor 1512 can execute the instructions to cause the system 100 to perform the methods described herein. The user interface 1516 interacts with the processor 1512 to cause transmission of electrical signals, e.g., at selected actuation frequencies, to the ablation unit 401b and via wires of the cabling extending through the catheter shaft 12a. These wires electrically couple the controller 1500 to the ablation unit 401b so that the controller 1500 can send electrical signals to the ablation unit 401b and cavitation/decalcification unit 401a, and receive electrical signals from the ablation unit 401b and cavitation/decalcification unit 401a. The processor 1512 can control the ablation unit excitation source 1518 to control the amplitude and timing of the electrical signals so as to control the power level and duration of the ultrasound signals emitted by ablation unit 401b. More generally, the controller 1500 can control one or more ultrasound treatment parameters that are used to perform sonication. In certain embodiments, the ablation unit excitation source 1518 can also detect electrical signals generated by ablation unit 401b and communicate such signals to the processor 1512 and/or circuitry of a control board. While the ablation unit excitation source 1518 in FIG. 15 is shown as being part of the controller, it is also possible that the ablation unit excitation source 1518 is external to the controller 1500 while still being controlled by the controller 1500, and more specifically, by the processor 1512 of the controller 1500.

The processor 1512 can control the decalcification unit excitation source 1518 to control the amplitude and timing of the electrical signals so as to control the power level and duration of the, e.g., ultrasound, signals emitted by decalcification unit 01b. More generally, the controller 1500 can control one or more decalcification parameters that are used to perform cavitation. In certain embodiments, the decalcification unit excitation source 1520 can also detect electrical signals generated by decalcification unit 401a and communicate such signals to the processor 1512 and/or circuitry of a control board. While the decalcification unit excitation source 1520 in FIG. 15 is shown as being part of the controller, it is also possible that the decalcification unit excitation source 1518 is external to the controller 1500 while still being controlled by the controller 1500, and more specifically, by the processor 1512 of the controller 1500. The user interface 1516 can include a touch screen and/or buttons, switches, etc., to allow for an operator (user) to enter patient data, select treatment parameters, view records stored on a storage/retrieval unit (not shown), and/or otherwise communicate with the processor 1512. The user interface 1516 can include a voice-activated mechanism to enter patient data or may be able to communicate with additional equipment so that control of the controller 1500 is through a separate user interface, such as a wired or wireless remote control. In some embodiments, the user interface 1516 is configured to receive operator-defined inputs, which can include, e.g., a duration of energy delivery, one or more other timing aspects of the energy delivery pulses (e.g., frequency, duty cycle, etc.), power, body lumen length, mode of operation, patient parameter, such as height and weight, and/or verification of artery diameter, or a combination thereof. Example modes of operation can include (but are not limited to): system initiation and set-up, catheter preparation, balloon inflation, verification of balloon apposition, pre-cooling, sonication, post-cooling, balloon deflation, and catheter removal, but are not limited thereto. In certain embodiments, the user interface 1516 provides a graphical user interface (GUI) that instructs a user how to properly operate the system 100. The user interface 1516 can also be used to display treatment data for review and/or download, as well as to allow for software updates, and/or the like.

The controller 1500 can also control a cooling fluid supply subsystem 1530, which can include a cartridge and reservoir or alternative types of fluid pumps, and/or the like. The cooling fluid supply subsystem 1530 is fluidically coupled to one or more fluid lumens 40 within catheter shaft 12a which in turn are fluidically coupled to the balloon 14. The cooling fluid supply subsystem 1530 can be configured to circulate a cooling liquid through the catheter 200 to the ablation unit 01b in the balloon 14. The cooling fluid supply subsystem 1530 may include elements such as a reservoir for holding the cooling fluid, pumps (e.g., syringes), a refrigerating coil, or the like for providing a supply of cooling fluid to the interior space of the balloon 14 at a controlled temperature, desirably at or below body temperature. The processor 1512 interfaces with the cooling fluid supply subsystem 1530 to control the flow of cooling fluid into and out of the balloon 14. For example, the processor 1512 can control motor control devices linked to drive motors associated with pumps for controlling the speed of operation of pumps (e.g., syringes). Such motor control devices can be used, for example, where the pumps are positive displacement pumps, such as peristaltic pumps. Alternatively, or additionally, a control circuit may include structures such as controllable valves connected in the fluid circuit for varying resistance of the circuit to fluid flow (not shown). The processor 1512 can monitor pressure measurements obtained by the pressure sensors (e.g., P1, P2 and P3) to monitor and control the cooling fluid through the catheter 200 and the balloon 14. The pressure sensors can also be used to determine if there is a blockage and/or a leak in the catheter 200. While the balloon 14 is in an inflated state, the pressure sensors can be used to maintain a desired pressure in the balloon 14, e.g., at a pressure of between 10 psi and 30 psi, but not limited thereto.

Advantages and benefits of embodiments of the present disclosure include, but are not limited to, removing calcification that may interfere with sonication and may affect arterial stiffness and elevations in blood pressure that would not otherwise be treated; and by providing a catheter device 200, the treatments would be significantly faster and less traumatic.

In an embodiment, a tissue treatment system for renal denervation includes a bubble generation device and an ablation device. The bubble generation device is configured to generate one or more bubbles to at least partially fragment a calcification of a calcified region of a renal artery. The ablation device is configured to ablate one or more nerves about, within, or surrounding the calcified region of the renal artery after the calcification has been at least partially fragmented.

In an embodiment, the tissue treatment system includes an imaging device configured to enable identification of the calcified region of the renal artery.

In an embodiment, the imaging device is configured to enable a determination of whether the calcification has been at least partially fragmented based on an output of the imaging device.

In an embodiment, the imaging device is an imaging transducer.

In an embodiment, the bubble generation device comprises at least two decalcification electrodes configured to generate the one or more bubbles.

In an embodiment, the bubble generation device comprises a decalcification transducer configured to generate the one or more bubbles.

In an embodiment, the tissue treatment system includes a non-transitory computer readable memory storing instructions, and one or more processors configured to execute the stored instructions to cause the tissue treatment system to perform operations. The operations include identifying the calcified region of the renal artery using an output of the imaging device, and energizing the decalcification transducer at a frequency range of 400 kHz to 3 MHz and at a power intensity of 1-50 W/cm² using 10 to 100 pulses 10 microsecond to 1 millisecond in duration and 20 milliseconds to 2 seconds apart.

In an embodiment, the tissue treatment system includes a first balloon, a non-transitory computer readable memory, and one or more processors. The first balloon surrounds at least the bubble generation device. The non-transitory computer readable memory stores instructions. The one or more processors are configured to execute the stored instructions to cause the tissue treatment system to: fill the first balloon with fluid such that the first balloon is in apposition with the renal artery, and maintain the first balloon at a constant pressure using a flow rate of 0 to 2 ml/min while the bubble generation device generates one or more bubbles.

In an embodiment, the bubble generation device is configured to generate the one or more bubbles by applying a voltage of 500 V/mm to 20 kV/mm across the at least two decalcification electrodes for a period of time.

In an embodiment, a tissue treatment system is configured to ablate one or more nerves about, within, or surrounding a calcified region of a renal artery. The tissue treatment system includes a non-transitory computer readable memory and one or more processors. The non-transitory computer readable memory stores instructions. The one or more processors are configured to execute the stored instructions to cause the tissue treatment system to: detect calcification within the renal artery in a target area, and lower a default acoustic entry power setting and increase a duration of ablation setting based on detecting calcification.

In an embodiment, the one or more processors lower the acoustic entry power by 30% and increases the treatment duration by 35-45%.

In an embodiment, the one or more processors additionally increase a flow rate by 5% or more based on detecting calcification.

In an embodiment, the tissue treatment system includes an ablation device, a non-transitory computer readable memory, and one or more processors. The ablation device is configured to ablate one or more nerves innervating a kidney. The non-transitory computer readable memory stores instructions. The one or more processors are configured to execute the stored instructions to cause the tissue treatment system to: detect a calcification score at one or more target locations along a main renal artery and at one or more target locations along at least one of an accessory renal artery or a renal artery branch; determine whether the calcification score is lower at one or more target locations along a main renal artery or at one or more target locations along at least one of an accessory renal artery or a renal artery branch; and prompt a user, using a graphic user interface, to perform an ablation at the one or more target locations along a main renal artery or, alternatively, at one or more target locations along at least one of an accessory renal artery or a renal artery branch based on whether the calcification score is lower at the one or more target locations along a main renal artery or at one or more target locations along at least one of an accessory renal artery or a renal artery branch.

In an embodiment, the one or more processors are further configured to execute the stored instructions to cause the tissue treatment system to: increase a default treatment duration, while maintaining a default flow rate and acoustic entry power level setting in order to create a lesion beginning about 1 mm from a lumen of the main renal artery to about 1 mm from the lumen of at least one of the accessory renal artery or a renal artery branch.

In an embodiment, the one or more processors are further configured to execute the stored instructions to cause the tissue treatment system to: decrease acoustic entry power below a default setting while increasing a treatment duration setting in order to create a lesion beginning about 1 mm from a lumen of the main renal artery to about 1 mm from the lumen of at least one of the accessory renal artery or a renal artery branch, while still compensating for calcification within the treated main renal artery or at least one of accessory renal artery or a renal artery branch.

In an embodiment, a method of renal denervation includes delivering a catheter to a calcified region of a body lumen wall. The catheter includes a proximal balloon, a middle balloon, and a distal balloon mounted on a catheter shaft. The catheter includes a laser fiber disposed on a surface of the middle balloon. The method includes generating, by the laser fiber, one or more bubbles to at least partially fragment the calcified region of the body lumen wall. The method includes ablating one or more nerves about, within, or surrounding the calcified region of the body lumen wall after the calcified region has been at least partially fragmented.

In an embodiment, the method includes inflating the proximal balloon and the distal balloon to substantially appose the body lumen wall to block calcified fragments from embolizing.

In an embodiment, the method includes inflating the middle balloon to position the laser fiber adjacent to the calcified region when generating the one or more bubbles.

In an embodiment, the method includes removing blocked calcified fragments with holes located on the catheter shaft.

In an embodiment, a method of renal denervation includes delivering a catheter to a calcified region of a body lumen wall. The catheter includes a barbell-shaped balloon mounted on a catheter shaft. The catheter includes a laser fiber disposed on a surface of a center region of the barbell-shaped balloon. The method includes inflating the barbell-shaped balloon such that the center region is longitudinally aligned with the calcified region, and the calcified region is longitudinally between a proximal region and a distal region of the barbell-shaped balloon. The method includes generating, by the laser fiber, one or more bubbles to at least partially fragment the calcified region of the body lumen wall. The method includes ablating one or more nerves about, within, or surrounding the calcified region of the body lumen wall after the calcified region has been at least partially fragmented.

In an embodiment, the method includes delivering a suction device through a space between the body lumen wall and the proximal region of the barbell-shaped balloon to remove fragments of the calcified region.

In an embodiment, the method includes inflating the proximal region and the distal region of the barbell-shaped balloon to substantially appose the body lumen wall to block calcified fragments from embolizing.

In an embodiment, the method includes inflating the center region to position the laser fiber adjacent to the calcified region when generating the one or more bubbles.

In an embodiment, a method of renal denervation includes delivering a catheter to a calcified region of a body lumen wall. The catheter includes a proximal balloon and a distal balloon mounted on a catheter shaft. The catheter includes a laser fiber disposed on a surface of the proximal balloon. The method includes generating, by the laser fiber, one or more bubbles to at least partially fragment the calcified region of the body lumen wall. The method includes ablating one or more nerves about, within, or surrounding the calcified region of the body lumen wall after the calcified region has been at least partially fragmented.

In an embodiment, the method includes inflating the distal balloon such that the distal balloon apposes the body lumen wall to block calcified fragments from embolizing.

In an embodiment, the method includes passing a scoop in a proximal direction to remove fragments of the calcified region.

In an embodiment, a computer program product includes program code portions for performing the operations of any of the above embodiments when the computer program is executed by a processing device. In an embodiment, a controller or a controller system includes memory that stores the computer program product.

In an embodiment, a method of renal denervation, includes identifying, using an imaging device, a calcified region of a renal artery. In an embodiment, the method includes blocking the renal artery. In an embodiment, the method includes delivering a laser fiber adjacent to the calcified region. In an embodiment, the method includes generating, by the laser fiber, one or more bubbles to at least partially fragment the calcified region. In an embodiment, the method includes removing fragments of the calcified region. In an embodiment, the method includes ablating a nerve surrounding the renal artery.

As used in the description and claims, the singular form “a”, “an” and “the” include both singular and plural references unless the context clearly dictates otherwise. For example, the term “unit” may include, and is contemplated to include, several units. At times, the claims and disclosure may include terms such as “a plurality,” “one or more,” or “at least one;” however, the absence of such terms is not intended to mean, and should not be interpreted to mean, that a plurality is not conceived. The term “calcified” and “calcium” both refer to calcification within the renal artery.

The term “about” or “approximately,” when used before a numerical designation or range (e.g., to define a length or pressure), indicates approximations which may vary by (+) or (−) 5%, 1% or 0.1%. All numerical ranges provided herein are inclusive of the stated start and end numbers. The term “substantially” indicates mostly (i.e., greater than 50%) or essentially all of a device, substance, or composition.

As used herein, the term “comprising” or “comprises” is intended to mean that the devices, systems, and methods include the recited elements, and may additionally include any other elements. “Consisting essentially of”′ shall mean that the devices, systems, and methods include the recited elements and exclude other elements of essential significance to the combination for the stated purpose. Thus, a system or method consisting essentially of the elements as defined herein would not exclude other materials, features, or operations that do not materially affect the basic and novel characteristic(s) of the claimed disclosure. “Consisting of”′ shall mean that the devices, systems, and methods include the recited elements and exclude anything more than a trivial or inconsequential element or operation. Embodiments defined by each of these transitional terms are within the scope of this disclosure.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A tissue treatment system for renal denervation, comprising:
a bubble generation device configured to generate one or more bubbles to at least partially fragment a calcification of a calcified region of a renal artery; and
an ablation device configured to ablate one or more nerves about, within, or surrounding the calcified region of the renal artery after the calcification has been at least partially fragmented.

2. The tissue treatment system of claim 1, further comprising an imaging device configured to enable identification of the calcified region of the renal artery.

3. The tissue treatment system of claim 2, wherein the imaging device is configured to enable a determination of whether the calcification has been at least partially fragmented based on an output of the imaging device.

4. The tissue treatment system of claim 2, wherein the imaging device is an imaging transducer.

5. The tissue treatment system of claim 1, wherein the bubble generation device comprises at least two decalcification electrodes configured to generate the one or more bubbles.

6. The tissue treatment system of claim 1, wherein the bubble generation device comprises a decalcification transducer configured to generate the one or more bubbles.

7. The tissue treatment system of claim 1, further comprising:
a non-transitory computer readable memory storing instructions; and
one or more processors configured to execute the stored instructions to cause the tissue treatment system to energize the decalcification transducer at a frequency range of 400 kHz to 3 MHz and at a power intensity of 1-50 W/cm² using 10 to 100 pulses 10 microsecond to 1 millisecond in duration and 20 milliseconds to 2 seconds apart.

8. The tissue treatment system claim 1, further comprising:
a first balloon surrounding at least the bubble generation device;
a non-transitory computer readable memory storing instructions; and
one or more processors configured to execute the stored instructions to cause the tissue treatment system to:
fill the first balloon with fluid such that the first balloon is in apposition with the renal artery, and
maintain the first balloon at a constant pressure using a flow rate of 0 to 2 ml/min while the bubble generation device generates one or more bubbles.

9. The tissue treatment system of claim, wherein the bubble generation device is configured to generate the one or more bubbles by applying a voltage of 500 V/mm to 20 kV/mm across the at least two decalcification electrodes for a period of time.

10. A tissue treatment system configured to ablate one or more nerves about, within, or surrounding a calcified region of a renal artery comprising:
a non-transitory computer readable memory storing instructions; and
one or more processors configured to execute the stored instructions to cause the tissue treatment system to:
detect calcification within the renal artery in a target area, and
lower a default acoustic entry power setting and increase a duration of ablation setting based on the detected calcification.

11. The tissue treatment system of claim 10, wherein the one or more processors are configured to lower the acoustic entry power by 30% and increases the treatment duration by 35-45%.

12. The tissue treatment system of claim 11, wherein the one or more processors are configured to additionally increase a flow rate by 5% or more based on the detected calcification.

13. A tissue treatment system comprising:
an ablation device configured to ablate one or more nerves innervating a kidney;
a non-transitory computer readable memory storing instructions; and
one or more processors configured to execute the stored instructions to cause the tissue treatment system to:
detect an amount of calcification at one or more target locations along a main renal artery and at one or more target locations along at least one of an accessory renal artery or a renal artery branch;
determine whether the calcification amount is lower at the one or more target locations along the main renal artery or at the one or more target locations along at least one of the accessory renal artery or the renal artery branch; and
prompt a user, using a graphic user interface, to perform an ablation at the one or more target locations along the main renal artery or, alternatively, at the one or more target locations along at least one of the accessory renal artery or the renal artery branch based on whether the calcification amount is lower at the one or more target locations along the main renal artery or at the one or more target locations along at least one of the accessory renal artery or the renal artery branch.

14. The tissue treatment system of claim 13, wherein the one or more processors are further configured to execute the stored instructions to cause the tissue treatment system to: increase a default treatment duration, while maintaining a default flow rate and acoustic entry power level setting in order to create a lesion beginning about 1 mm from a lumen of the main renal artery to about 1 mm from the lumen of at least one of the accessory renal artery or the renal artery branch.

15. The tissue treatment system of claim 13, wherein the one or more processors are further configured to execute the stored instructions to cause the tissue treatment system to decrease acoustic entry power below a default setting while increasing a treatment duration setting in order to create a lesion beginning about 1 mm from a lumen of the main renal artery to about 1 mm from the lumen of at least one of the accessory renal artery or the renal artery branch, while still compensating for calcification within the treated main renal artery or at least one of the accessory renal artery or the renal artery branch.

16. A method of renal denervation, comprising:
delivering a catheter to a calcified region of a body lumen wall, wherein the catheter includes a proximal balloon, a middle balloon, and a distal balloon mounted on a catheter shaft, and wherein the catheter includes a laser fiber disposed on a surface of the middle balloon;
generating, by the laser fiber, one or more bubbles to at least partially fragment the calcified region of the body lumen wall; and
ablating one or more nerves about, within, or surrounding the calcified region of the body lumen wall after the calcified region has been at least partially fragmented.

17. The method of claim 16, further comprising inflating the proximal balloon and the distal balloon to substantially appose the body lumen wall to block calcified fragments from embolizing.

18. The method of claim 16, further comprising inflating the middle balloon to position the laser fiber adjacent to the calcified region when generating the one or more bubbles.

19. The method of any of claim 16, further comprising removing blocked calcified fragments with holes located on the catheter shaft.

20. A method of renal denervation, comprising:
delivering a catheter to a calcified region of a body lumen wall, wherein the catheter includes a barbell-shaped balloon mounted on a catheter shaft, and wherein the catheter includes a laser fiber disposed on a surface of a center region of the barbell-shaped balloon;
inflating the barbell-shaped balloon such that the center region is longitudinally aligned with the calcified region, and the calcified region is longitudinally between a proximal region and a distal region of the barbell-shaped balloon;
generating, by the laser fiber, one or more bubbles to at least partially fragment the calcified region of the body lumen wall; and
ablating one or more nerves about, within, or surrounding the calcified region of the body lumen wall after the calcified region has been at least partially fragmented.

21. The method of claim 20, further comprising delivering a suction device through a space between the body lumen wall and the proximal region of the barbell-shaped balloon to remove fragments of the calcified region.

22. The method of claim 20, further comprising inflating the proximal region and the distal region of the barbell-shaped balloon to substantially appose the body lumen wall to block calcified fragments from embolizing.

23. The method of claim 22, further comprising inflating the center region to position the laser fiber adjacent to the calcified region when generating the one or more bubbles.

24. A method of renal denervation, comprising:
delivering a catheter to a calcified region of a body lumen wall, wherein the catheter includes a proximal balloon and a distal balloon mounted on a catheter shaft, and wherein the catheter includes a laser fiber disposed on a surface of the proximal balloon;
generating, by the laser fiber, one or more bubbles to at least partially fragment the calcified region of the body lumen wall; and
ablating one or more nerves about, within, or surrounding the calcified region of the body lumen wall after the calcified region has been at least partially fragmented.

25. The method of claim 24, further comprising inflating the distal balloon such that the distal balloon apposes the body lumen wall to block calcified fragments from embolizing.

26. The method of claim 24, further comprising passing a scoop in a proximal direction to remove fragments of the calcified region.

27. A computer program product comprising program code portions for performing the operations of claim 16 when the computer program is executed by a processing device.

28. A controller or a controller system comprising memory that stores the computer program product of claim 27.
